SEQUENCE LISTING (3 sequences)

SEQ ID NO: 1 - protein, 147 aa, Homo sapiens
MPALPEDGGSGAFPPGHFKDPKRLYCKNGGFFLRIHPDGRVDGVREKSDPHIKLQLQAEERGVVSIKGVSANRYLAMKEDGRLLASKSVTDECFFFERLESNNYNTYRSRKYTSWYVALKRTGQYKLGSKTGPGQKAILFLPMSAKS

SEQ ID NO: 2 - DNA, 444 bp, Homo sapiens
  CDS (1)..(441)
atgccagcattgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcactgaaacgaactgggcagtataaacttggatccaaaacaggacctgggcagaaagctatactttttcttccaatgtctgctaagagctga

SEQ ID NO: 3 - protein, 147 aa, Homo sapiens
  MOD_RES (70)..(70): Gly, Thr, Tyr, Asp, or Glu
MPALPEDGGSGAFPPGHFKDPKRLYCKNGGFFLRIHPDGRVDGVREKSDPHIKLQLQAEERGVVSIKGVXANRYLAMKEDGRLLASKXVTDECFFFERLESNNYNTYRSRKYTSWYVALKRTGQYKLGSKTGPGQKAILFLPMSAKS

(19) United States
(12) Patent Application Publication
(10) Pub. No.: US 2009/0111748 A1
(43) Pub. Date: Apr. 30, 2009

(54) FIBROBLAST GROWTH FACTOR-2 PROMOTES NEUROGENESIS AND NEUROPROTECTION AND PROLONGS SURVIVAL IN HUNTINGTON'S DISEASE

(76) Inventors: Lisa M. Ellerby, Novato, CA (US); David A. Greenberg, Sonoma, CA (US); Julie Andersen, Novato, CA (US); Kunlin Jin, Novato, CA (US)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 11/996,178

(22) PCT Filed: Jul. 21, 2006
(86) PCT No.: PCT/US06/28680

§ 371 Date: Jul. 15, 2008

Related U.S. Application Data
(60) Provisional application No. 60/701,752, filed on Jul. 21, 2005.

(51) Int. Cl. A61K 38/18

(52) U.S. Cl. 514/12

(57) ABSTRACT

This invention pertains to the discovery that fibroblast growth factor 2 (FGF2) stimulates neurogenesis, induces migration of newborn cells into the striatum and cortex, is neuroprotective, and significantly extends the lifespan mammals suffering from neurodegenerative conditions (e.g., Huntington's disease, Parkinson's disease, etc.). In certain embodiments this invention provides a method of promoting neurogenesis, neuroprotection and/or survival in a mammal having a neurodegenerative disease by upregulating expression or availability of endogenous fibroblast growth factor 2 (FGF2) in said mammal; and/or administering FGF2 or an FGF2 mutein to the mammal in an amount sufficient to promote neurogenesis, neuroprotection and/or survival of the mammal.

### CROSS-REFERENCE TO RELATED APPLICATIONS

This application claims benefit of and priority to U.S. Ser. No. 60/701,752, filed on Jul. 21, 2005, which is incorporated herein by reference for all purposes.

### STATEMENT AS TO RIGHTS TO INVENTIONS MADE UNDER FEDERALLY SPONSORED RESEARCH AND DEVELOPMENT

This work was supported, in part, by grants NS44921, AG21980, and NS40251 from the National Institutes of Health. The Government of the United States of America has certain rights in this invention.

### FIELD OF THE INVENTION

This invention pertains to the treatment of neurodegenerative diseases (e.g., Huntington's disease, Parkinson's disease, etc.). In particular, this invention pertains to the discovery that fibroblast growth factor 2 (FGF2) can promoting neurogenesis, neuroprotection and/or survival in a mammal having a neurodegenerative disease.

### BACKGROUND OF THE INVENTION

A number of diseases are characterized by progressive neural degeneration. Huntington's disease (ID), for example, is a progressive and fatal neurological disorder caused by a polyglutamine expansion in the N-terminus of the protein huntingtin (Htt).

Expansions greater than 36 glutamines typically cause the disease. There is currently no treatment to delay the appearance or progression of the disease. HD is characterized by a dramatic loss of neurons in the striatum and cerebral cortex, resulting in chorea, dementia and early death. Multiple molecular pathways are involved in the pathophysiology of HD. Disease initiation and progression are thought to involve a conformational change in the Htt protein due to the polyglutamine expansion (Perutz (1999) Trends Biochem Sci 24: 58-63), altered protein-protein interactions (Wanker et al. (1997) Hum Mol Genet 6: 487-495; Kalchman et al. (1997) Nat Genet 16: 44-53; Harjes and Wanker (2003) Trends Biochem Sci 28, 425-33), abnormal protein aggregation (Davies et al. (1997) Cell 90: 537-548) and proteolysis, leading to transcriptional dysregulation (Goldberg et al. (1996) Nat Genet 13: 442-449; Nucifora et al. (2001) Science 291: 2423-2428; Zuccato et al. (2003) Nat Genet 35: 76-783), excitotoxicity (Ferrante et al. (1985) Science 230: 561-563; Browne et al. (1997) Ann Neurol 41: 646-653; Zeron et al. (2001) Mol Cell Neurosci 17: 41-53) and mitochondrial dysfunction (Panov et al. (2002) Nat Neurosci 5: 731-736), culminating in extensive loss of neurons in the striatum and cerebral cortex (Ferrante et al. (1985) Science 230: 561-563). The precise cause of neuronal cell death and the relative contributions of the various above-mentioned abnormalities to this process are not known.

Parkinson's disease (PD) is the second most frequently occurring neurodegenerative disorder after Alzheimer's disease (AD), affecting about 1% of the population over the age of 50 in the North America (Formo (1996) J Neuropathol Exp Neurol 55:259-272; Lang and Lozano (1998) N Engl J Med 339:1044-1053). Despite progress in understanding molecular mechanisms in PD, fully effective treatment remains elusive. One new potential strategy for replacing midbrain dopaminergic neurons in PD is based on endogenous neuroproliferation in the rostral subventricular zone (SVZ) of the lateral ventricles and the subgranular zone (SGZ) of the dentate gyrus (DG). Neurogenesis is increased in these regions in certain neurological disorders including Alzheimer's disease (Jin et al. (2004a) Proc. Natl. Acad. Sci., USA, 101:343-347), Huntington's disease (HD) (Curtis et al. (2003) Proc. Natl. Acad. Sci., USA, 100: 9023-9027) and stroke (Jin et al. (2001) Proc. Natl. Acad. Sci., USA, 98:4710-4715).

### SUMMARY OF THE INVENTION

This invention pertains to the discovery that FGF2 treatment can reduce neuronal loss, increase neurogenesis and improve functional outcome in various neurodegenerative conditions (e.g. Huntington's disease, parkinson's disease, etc.). We show that FGF2 stimulates neurogenesis, induces migration of newborn cells into the striatum and cortex, is neuroprotective, and significantly extends the lifespan of HD transgenic R6/2 mice. In addition, we show that Fibroblast growth factor-2 (FGF2), which increased the number of BrdU/Dcx-immunopositive cells in the SN of MPTP-treated mice.

Thus, in certain embodiments this invention provides for methods of promoting neurogenesis, neuroprotection and/or survival in a mammal having a disease characterized by neural degeneration. The methods typically involve administering FGF2 or an FGF2 mutein to the mammal in an amount sufficient to promote neurogenesis, neuroprotection and/or survival of the mammal. In certain embodiments the FGF2 is a human FGF2 or a human FGF2 mutein. In certain embodiments the FGF2 or FGF2 mutein is a recombinantly expressed FGF2. In various embodiments the FGF2 is an isolated FGF2. In various embodiments the FGF2 mutein is a cysteine depleted FGF mutein. In various embodiments the mammal is a human having or at risk for a neurodegenerative disease (e.g., familial amyotrophic lateral sclerosis (FALS), sporadic amyotrophic lateral sclerosis (ALS), familial and sporadic Parkinson's disease, Huntington's disease, familial and sporadic Alzheimer's disease, olivopontocerebellar atrophy, multiple system atrophy, progressive supranuclear palsy, diffuse lewy body disease, corticodentatonigral degeneration, progressive familial myoclonic epilepsy, strionigral degeneration, torsion dystonia, familial tremor, Gilles de la Tourette syndrome, Hallervorden-Spatz disease, and the like). The FGF2 or FGF2 mutein can be administered systemically or, in certain embodiments, directly to the brain. In various embodiments the administration is subcutaneous or intraperitoneal. In certain embodiments the administration is by administration of an expression vector harboring a human FGF2 or FGF2 mutein cDNA. In various embodiments the expression vector is a retroviral expression vector.

This invention also provides for the use of an FGF2 or FGF2 mutein in the manufacture of a medicament for the treatment or prophylaxis of a neurodegenerative disease.

This invention also provides a method of promoting neurogenesis, neuroprotection and/or survival in a mammal having a disease characterized by neural degeneration (e.g., familial amyotrophic lateral sclerosis (FALS), sporadic amyotrophic lateral sclerosis (ALS), familial and sporadic Parkinson's disease, Huntington's disease, familial and sporadic Alzheimer's disease, olivopontocerebellar atrophy, multiple system atrophy, progressive supranuclear palsy, diffuse lewy body disease, corticodentatonigral degeneration, progressive familial myoclonic epilepsy, strionigral degeneration, torsion dystonia, familial tremor, Gilles de la Tourefte syndrome, Hallervorden-Spatz disease, and the like), where the method involves upregulating expression or availability of endogenous fibroblast growth factor 2 (FGF2) in the mammal. In certain embodiments the FGF2 expression is increased by radiation treatment and/or by treatment with an antidepressant (e.g., tricyclics and/or SSRIs), and/or by a β2-adrenergic receptor agonist. In certain embodiments the mammal is a human having or at risk for the disease. The therapeutic agent(s) can be administered systemically or, in certain embodiments, directly to the brain. In various embodiments the administration is subcutaneous or intraperitoneal.

In certain embodiments, the subjects are humans not undergoing treatment with antidepressants.

### DEFINITIONS

The terms “polypeptide”, “peptide” and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers. It will also be appreciated in addition to the peptide sequences expressly illustrated herein, in certain embodiments, this invention also contemplates retro-, inverse (inverso-), and retro-inverso forms of each of these peptides. In retro forms, the direction of the sequence is reversed. In inverse forms, the chirality of the constituent amino acids is reversed (i.e., L form amino acids become D form amino acids and D form amino acids become L form amino acids). In the retro-inverso form, both the order and the chirality of the amino acids is reversed.

The terms “Fibroblast Growth Factor 2” and “FGF2” are used interchangeably. FGF2 also known as basic fibroblast growth factor (bFGF) is a heparin binding growth factor which stimulates the proliferation of a wide variety of cells including mesenchymal, neuroectodermal and endothelial cells. bFGF also exerts a potent angiogenic activity in vivo. Human bFGF is a 17.2 kDa protein containing 154 amino acid residues. bFGF synergizes with the BMP antagonist noggin to sustain undifferentiated proliferation of human embryonic stem (hES) cells under feeder-free conditions (see, e.g., Xu, et al. (2005) Nature Methods 2(3): 185-190). The term FGF2, as used herein, includes both full-length FGF2 as well as truncated FGF2 molecules that possess identical or essentially the same biological activity as full-length human FGF2.

The term “conservative substitution” is used herein to refer to replacement of amino acids in a protein with different amino acids that do not substantially change the functional properties of the protein. Thus, for example, a polar amino acid might be substituted for a polar amino acid, a non-polar amino acid for a non-polar amino acid, and so forth. The following six groups each contain amino acids that are conservative substitutions for one another: 1) Glycine, Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W).

### BRIEF DESCRIPTION OF THE DRAWINGS

FIG. 1 illustrates an FGF2 mutein (SEQ ID NO: 1) having a cysteine to serine mutation at two sites. In various embodiments either or both of the sites can be mutated to serine or to another amino acid (e.g., an amino acid having an uncharged polar R group, such as glycine, threonine, tyrosine, asparagine, glutamine, and the like).

FIGS. 2A, 2B, and 2C show that FGF-2 treatment enhances neurogenesis in HD transgenic R6/2. FIGS. 2A and 2B: HD transgenic R6/2 and wild-type control mice were given intraperitoneal BrdU for 3 days, treated with subcutaneous vehicle (PBS) or FGF-2 (three weeks), and killed 24 hours later. Immunocytochemistry showed a modest increase in the number of BrdU-labeled cells (brown) in SVZ of PBS-treated HD transgenic R6/2 compared to wild-type mice. FGF-2 enhanced BrdU labeling slightly in wild-type and markedly in HD transgenic R6/2 mice. FIG. 2C: Immunocytochemistry with anti-DCX antibody showed an increase in the number of DCX-expressing (new) neurons in FGF-compared to PBS-treated HD transgenic R6/2 mice. Inset in shows a DCX migrating cell. Data are representative fields from at least three experiments per panel (A,C) or mean±SEM, n=3. *p<0.05; **p<0.01 relative to PBS-treated mice.

FIGS. 3A-3E show that FGF-2 treatment generates DARPP-32-expressing striatal and NeuN-expressing cortical neurons. (FIGS. 3A and 3B) DARPP-32 (green) and DCX (red) were co-expressed in striatal (Str) neurons (Panel A) and NeuN (green) and DCX (red) co-localized in cortical (Ctx) neurons (Panel B) from FGF-2-treated HD transgenic R6/2 mice. Littermate control mice treated with FGF-2 did not have DCX and DARPP-32 co-expressed. Scale bar 10 m. (FIG. 3C) Mice received stereotaxic injection ([anterior posterior −0.3 mm, lateral 1.7 mm, depth 3.5 mm]) into the globus pallidus with retrograde tracer Alexa Fluoro 488 (panel a). Retrograde tracer Alexa Fluoro 488 (panel B,C) could be detected in the caudate via retrograde transport. No diffusion of Alexa Fluoro 488 was detected from the globus pallidus. (FIG. 3D) Alexa Fluoro 488 (green), DCX (red) and NeuN (blue) were coexpressed in FGF-2 treated mice demonstrating extending fibers into the pallidal targets. Scale bar 10 m. (FIG. 3E) Confocal images of Alexa Fluoro 488 (green) and BrdU (red) in the striata of FGF-2 treated HD transgenic R6/2 mice, shown as both single optical sections and orthogonal views in the xz and yz planes, to confirm that some BrdU cells were positive for retrograde tracer, shown as both single optical sections and orthogonal views in the xz and yz planes (left panel). Confocal images of DARPP-32 (green) and BrdU (red) in the striata of FGF-2 treated HD transgenic R6/2 mice, shown as both single optical sections and orthogonal views in the xz and yz planes, to confirm that some BrdU cells were positive for DARPP-32, shown as both single optical sections and orthogonal views in the xz and yz planes (right panel).

FIGS. 4A and 4B show that FGF-2 prolongs survival and improves rotarod performance in HD transgenic R6/2 mice. FIG. 4A: HD transgenic R6/2 mice were given PBS (HD untreated) or FGF-2 (HD+FGF-2) beginning at 59 days of age (arrow) and survival was plotted. FGF-2 increased survival, as described in Results (n=10, *p<0.05). FIG. 4B: Motor performance was evaluated with a rotarod apparatus in 11- and 13-week old littermate controls (NonTg) and in HD transgenic R6/2 mice (HD) treated with treated PBS or FGF-2. The total time spent on the rod during a 5-min period (latency) was recorded. Values are the means ±SEM (=10 per group); **p<0.01 compared to PBS.

FIGS. 5A-5D show that FGF-2 is neuroprotective in HD striatal neuron cultures and does not increase BDNF or CNTF levels in HD transgenic R6/2 mice. FIG. 5A: Immortalized striatal neurons expressing wild type Htt ( ) or a knocked-in HD mutation with 111 polyglutamine repeats ( ) were subjected to serum withdrawal and cellular viability was assessed with WST-1 assay. **p<0.01 compared to untreated cultures (ANOVA, n=3). FIG. 5B: Electroporation with a GFP-expressing vector resulted in greater than 50% transfection efficiency in primary cultures of striatal neurons, as shown by immunostaining for GFP (left). Striatal neurons transfected with a mutant Htt147Q (1-110) construct showed extensive cell death (72 hours, center), which was rescued by treatment with FGF-2 (right). Cultures shown in the center and right panels were immunostained with monoclonal anti-Htt 2170 (Chemicon; 1:100). Nuclei were counterstained with DAPI (blue). FIG. 5C: Striatal neurons were transfected with Htt23Q (1-110), Htt138Q-GFP or Htt147Q (1-110) and cell viability was assessed using calcein-AM and ethidium homodimer-1 (LIVE/DEAD kit, Molecular Probes) at 48 hours. Data shown are mean values±SEM, (n=3-5). *p<0.05 compared to untreated cultures (ANOVA, n=3). FIG. 5D: Western blot analysis of BDNF and CNTF levels of striatal lysates from 11-week old littermate controls (WT) and HD transgenic R6/2 mice (HD) treated with PBS or FGF-2 for three weeks, with anti-actin used as a control for differences in protein loading.

FIGS. 6A-6C show histopathological evidence of neuroprotection by FGF-2 in 11-week old HD transgenic R6/2 mice. FIG. 6A: Ubiquitin immunohistochemistry (brown) in PBS- and FGF-2-treated littermate control (WT) and HD transgenic R6/2 mice showed ubiquitin immunoreactivity in PBS-treated HD mice, but not in WT or FGF-2 treated HD mice (top two rows). Htt immunohistochemistry (brown) in neostriatum (Str) and cortex (Ctx) showed Htt-immunoreactive aggregates (arrows) in PBS- but not in FGF-2-treated HD transgenic R6/2 mice (bottom two rows). FIG. 6B: Immunohistochemistry with a polyglutamine antibody that recognizes mutant human Htt and Htt-containing aggregates showed abundant aggregates in striatum (not shown) and neocortex of PBS-treated (left) but not FGF-2-treated (right) HD transgenic R6/2 mice. FIG. 6C: CB1 caniabinoid receptor and DARPP-32 immunoreactivity in PBS- and FGF-2-treated littermate control (WT) and HD transgenic R6/2 (HD) mice showed that both CB 1 and DARPP-32 were depleted from the affected striatum and restored by FGF-2 treatment. The counterstain is Hematoxylin.

FIG. 7 shows a Western blot analysis of CB 1 cannabinoid receptor and DARPP-32 levels in striatal lysates from 11-week old littermate controls (WT) and HD transgenic R6/2 mice (HD) treated with PBS or FGF-2 for three weeks, with anti-actin used as a control for differences.

FIGS. 8A-8D illustrate neurogenesis in DG and SVZ of MPTP-treated mice. Representative sections showing BrdU-positive cells in the DG and SVZ (FIG. 8A) in control and MPTP-treated mice, and quantitative comparison of BrdU-positive cells in the DG (FIG. 8B) and SVZ (FIG. 8C) at various time points after MPTP administration (white bars, saline-treated; black bars, MPTP-treated). Mean±S.E., n=5. *p<0.01, **p<0.001, significantly from saline-treated. FIG. 8: Relationship between BrdU incorporation and Dcx expression in DG and SVZ. Colocalization (yellow) of BrdU (red) and Dcx (green) is increased in sections through the DG and SVZ of MPTP-compared to saline-treated mice at 2 weeks following the last MPTP injection.

FIGS. 9A and 9B illustrate neurogenesis in striatum of MPTP-treated mice. FIG. 9 A: Following acute MPTP treatment, Dcx immunoreactive newborn neurons (brown) are observed not only in the SVZ (top arrow in each panel) but also in the adjacent the striatum (bottom arrow in two right panels). FIG. 9B: Relationship between BrdU (red) and cell type-specific markers (green) in SN. Colocalization (yellow, white arrows) of BrdU (red) and PSA-NCAM (green), but not other cell-type markers, in SN after MPTP treatment. Original magnification, ×60.

FIGS. 10A and 10B show that fibroblast growth factor-2 (FGF2) promotes neurogenesis in SN. FIG. 10A: Quantitation of BrdU-positive profiles from saline (white bars)- or MPTP (black bars)-treated mice. Mean±S.E., n=4 *, p<0.01, significantly from saline group; #, p<0.05, significantly from vehicle plus MPTP group. FIG. 10B: Colocalization (white arrow) of BrdU (blue) and Dcx (green), but not TH (red) immunoreactivity in SN after MPTP treatment. Scale bar, 10 μm.

FIG. 11 shows co-localization of BrdU (blue), Dcx (green) and TH (red) in the SN of MPTP-induced adult mouse brain following FGF-2 administration. Scale bar, 10 μm.

### DETAILED DESCRIPTION

This invention pertains to the surprising discovery that fibroblast growth factor 2 (FGF2) can induce migration of newborn cells into the striatum and cortex, is neuroprotective, and significantly extends the lifespan of mammals subject to subject to various pathologies characterized by neurodegeneration (e.g., Huntington's disease, Parkinson's disease, and the like). In this regard, it is noted that unlike many agents, FGF-2 can be administered systemically and cross the blood-brain barrier to produce cerebral effects (Wagner et al. (1999) J Neurosci 19: 6006-6016; Jin et al. (2003) Ann Neurol 53: 405-409), obviating the requirement for more complex or invasive modes of delivery.

Thus, in certain embodiments, this invention contemplates administering FGF2, FGF2 muteins, or FGF2 mimetics to mammalian subjects diagnosed as having or being at risk for one or more pathologies characterized by neural degeneration. Such pathologies include, but are not limited to familial amyotrophic lateral sclerosis (FALS), sporadic amyotrophic lateral sclerosis (ALS), familial and sporadic Parkinson's disease, Huntington's disease, familial and sporadic Alzheimer's disease, olivopontocerebellar atrophy, multiple system atrophy, progressive supranuclear palsy, diffuse lewy body disease, corticodentatonigral degeneration, progressive familial myoclonic epilepsy, strionigral degeneration, torsion dystonia, familial tremor, Gilles de la Tourette syndrome, and Hallervorden-Spatz disease, and the like. In certain embodiments in certain embodiments, this invention contemplates administering FGF2, FGF2 muteins, or FGF2 mimetics to mammalian subjects diagnosed as having or being at risk for Parkinson's disease and/or Huntington's disease.

Exogenous FGF2, FGF2 muteins, and/or FGF2 mimetics can be administered and/or, in certain embodiments, the expression of endogenous FGF2 can be increased, e.g. by administration of drugs that increase endogenous FGF2 expression or availability. In certain embodiments FGF2 is administered by administering a vector carrying a nucleic acid (e.g. a DNA or RNA) that encodes an FGF2 and/or FGF2 mutein to the subject.

While this invention is discussed with respect to “native” FGF2, in certain embodiments, the methods can be practiced with FGF2 muteins and/or mimetics. Such muteins include, but are not limited to, those produced by replacing one or more of the “native” amino acid residues in an FGF2 amino acid sequence with a different amino acid, e.g., as described herein. Typically, such muteins will have conservative amino acid changes. For example, a useful mutein may include a serine residue in place of a cysteine residue, and so forth.

### I. FGF2 and FGF2 Muteins.

As indicated above, in certain embodiments, this invention pertains to the use of FGF2 and/or FGF2 muteins to mitigate one or more symptoms of a pathology characterized by neural degeneration. The FGF2 or FGF2 muteins act to promoting neurogenesis, neuroprotection and/or survival in a mammal at risk for or subject to a neurodegenerative conditions.

In certain embodiments the FGF2 can be an isolated naturally-occurring FGF2 or a recombinantly expressed FGF2. The FGF2 can be a full-length FGF2 or an FGF2 fragment that possess the characteristic activity (e.g., angiogenic activity) of full-length FGF2. In certain embodiments an FGF2 mutein is utilized.

Human FGF2 is commercially available (see, e.g., Chemicon International, GF003-AF). In addition, methods for making recombinant FGF2 and/or FGF2 muteins are well-known in the art. For example, the recombinant expression of bovine FGF2 is described in detail in U.S. Pat. No. 5,155,214, which is incorporated herein by reference. As disclosed in the '214 patent, a DNA encoding the FGF2 polypeptide is inserted into a cloning vector, such as pBR322, pMB9, Col E1, pCR1, RP4 or lambda-phage, and the cloning vector is used to transform either a eukaryotic or prokaryotic cell, whereby the transformed cell expresses the FGF2. In one embodiment, the host cell is a yeast cell, such as Saccharomyces cerevisiae. Using the methods described therein or other methods for recombinant expression of proteins known to those of skill in the art, a human FGF2 (see, e.g., SEQ ID NO:2) or FGF2 muteins (see, e.g., SEQ ID NO: 1) can readily be produced.

FGF2 as used herein contemplates full length FGF2 as well as truncated FGF2 and FGF2 fragments that possesses the characteristic activity (e.g., angiogenic activity) of FGF2. In various embodiments such an angiogenically active fragment comprises a fragment of FGF-2 that has at least about 70%, preferably at least about 80%, and more preferably at least about 90% or 95% of the 146 residues of human FGF2 that retains at least 50%, preferably at least 80%, more preferably at least 90%, and most preferably at least 95% or 99% of the angiogenic activity of human FGF2.

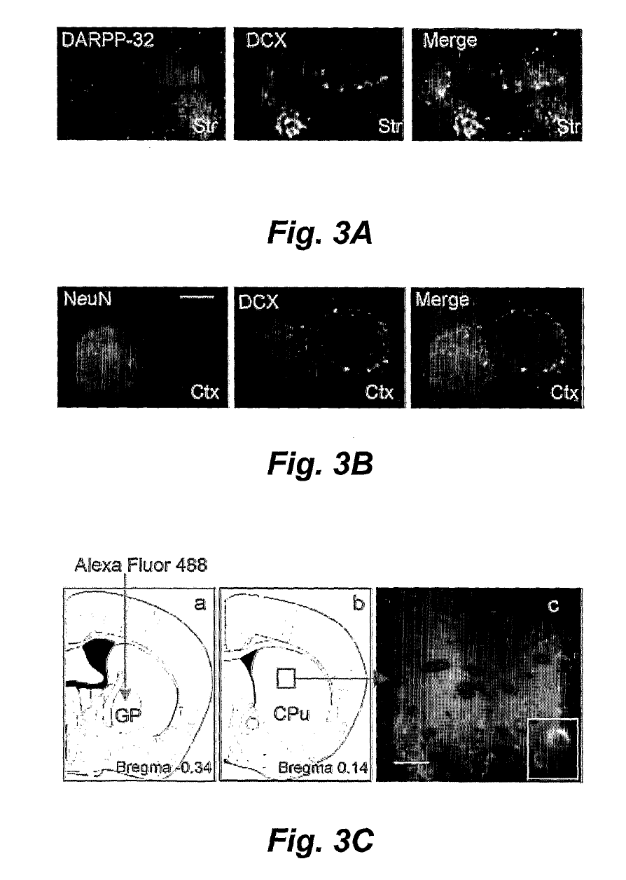

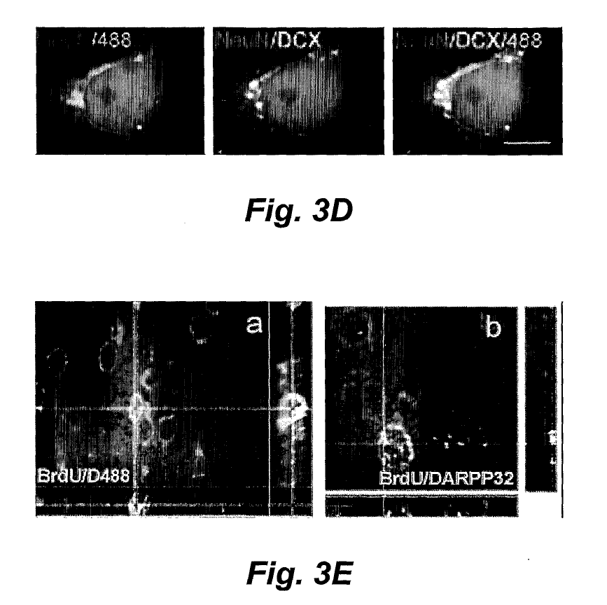

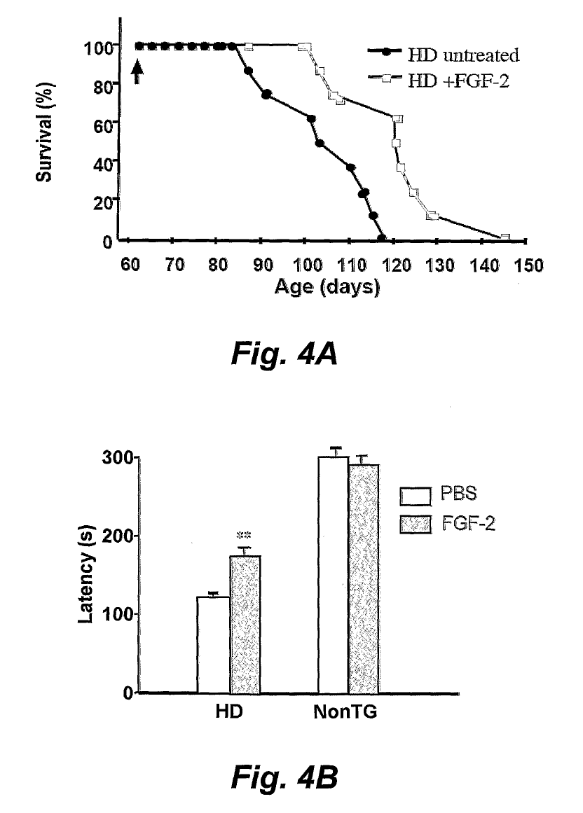

For activity, the FGF2 fragment comprise at least one cell binding site, preferably at least two cell binding sites and at least one of the two heparin binding sites. The two putative cell binding sites of human FGF-2 occur at residue positions 36-39 and 77-81 (see, e.g., Yoshida, et al., (1987) Proc. Natl. Acad. Sci., USA, 84:7305-7309 at FIG. 3). The two putative heparin binding sites of hFGF-2 occur at residue positions 18-22 and 107-111 (Id.).

Fragments of bovine FGF2 (bFGF2) that are known to have angiogenic activity are bFGF2 (24-120)-OH and bFGF2 (30-110)-NH₂ (see, e.g., U.S. Pat. No. 5,155,214 which is incorporated herein by reference). These latter fragments retain both of the cell binding portions of FGF2) and one of the heparin binding segments (residues 107-111). Accordingly, certain angiogenically active fragments of human FGF2 typically encompass those terminally truncated fragments of FGF-2 that have at least residues that correspond to residues 30-110 of bFGF2 more typically, at least residues that correspond to residues 18-146 of bFGF2.

The two cell binding sites for FGF2 are approximately at residue positions 36-39 and 77-81 thereof, and the two heparin binding sites are approximately at residue positions 18-22 and 107-111 thereof.

It is also known that N-terminal truncations, e.g. up to the first 30 amino acids, preferably up to the first 12, 15, or 24 amino acids, more preferably up to the first 5, 6, 7, 8, or 9 amino acids do not substantially reduce the activity of FGF2. Thus, in certain embodiments, deletion of one or more of these amino acids is contemplated.

In this regard, it is well known in the art that N-terminal truncations of bovine FGF2 do not eliminate its activity in cows. In particular, the art discloses several naturally occurring and biologically active fragments of the FGF-2 that have N-terminal truncations. An active and truncated bFGF-2 having residues 12-146 (relative to SEQ ID NO:2 in U.S. Pat. No. 6,440,934, which is incorporated by reference) was found in bovine liver and another active and truncated bFGF-2, having residues 16-146 (relative to SEQ ID NO:2 in U.S. Pat. No. 6,440,934) was found in the bovine kidney, adrenal glands and testes (see, e.g., U.S. Pat. No. 5,155,214, which is incorporated herein by reference, and Ueno et al. (1986) Biochem and Biophys Res. Comm., 138: 580-588). Likewise, other fragments of the bFGF2 (relative to SEQ ID NO:2 in U.S. Pat. No. 6,440,934) that are known to have FGF activity are bFGF2 (24-120)-OH and bFGF2 (30-110)-NH₂ (see, e.g., U.S. Pat. No. 5,155,214). These latter fragments retain both of the cell binding portions of FGF2 and one of the heparin binding segments (residues 107-111). Corresponding human FGF2 fragments are expected to have similar activity.

Accordingly, in certain embodiments the angiogenically active fragments of FGF2 typically encompass those terminally truncated fragments of FGF2 that have at least residues that correspond to residues 30-110 of bFGF-2 ((relative to SEQ ID NO:2 in U.S. Pat. No. 6,440,934) more typically, at least residues that correspond to residues 18-146 of bGF-2 ((relative to SEQ ID NO:2 in U.S. Pat. No. 6,440,934).

In addition this invention contemplates FGF2 muteins and FGF2 fragment muteins that posses the characteristic activity of FGF2. Typical muteins for use in this invention (e.g., angiogenically active . . . mutein(s)) include an isolated and/or purified recombinant protein or polypeptide that has at least 65%, preferably at least 75%, more preferably at least 80% or 90%, and most preferably at least 95% or at least 98% sequence identity (homology) to any naturally occurring FGF-, as determined by the Smith-Waterman homology search algorithm (see, e.g., Smith-Waterman et al. (1997) Meth. Mol. Biol. 70:173-187) as implemented in MSPRCH program (Oxford Molecular) using an affine gap search with the following search parameters: gap open penalty of 12, and gap extension penalty of 1, and that retains at least 50% or 75%, more preferably at least 80% or 85%, and most preferably at least 90%, 95%, or 98% of the angiogenic activity of the naturally occurring FGF2 with which it has said at least 65% sequence identity.

Other well-known and routinely used homology/identity scanning algorithm programs include Pearson and Lipman (1988) Proc. Natl. Acad. Sci., USA, 85:2444-2448; Lipman and Pearson (1985) Science, 222:1435; Devereaux et al. (1984) Nuc. Acids Res., 12: 387-395; or the BLASTP, BLASTN or BLASTX algorithms of Altschul et al. (1990) Mol. Biol., 215: 403-410. Computerized programs using these algorithms are also available and include, but are not limited to: GAP, BESTFIT, BLAST, FASTA and TFASTA, which are commercially available from the Genetics Computing Group (GCG) package, Version 8, Madison Wis., USA; and CLUSTAL in the PC/Gene program by Intellegenetics, Mountain View, Calif. Preferably, the percentage of sequence identity is determined by using the default parameters determined by the program.

The phrase “sequence identity,” as used herein, is intended to refer to the percentage of the same amino acids that are found similarly positioned within the mutein sequence when a specified, contiguous segment of the amino acid sequence of the mutein is aligned and compared to the amino acid sequence of the naturally occurring FGF2.

When considering the percentage of amino acid sequence identity in the mutein, some amino acid residue positions may differ from the reference protein as a result of conservative amino acid substitutions, which do not affect the properties of the protein or protein function. In these instances, the percentage of sequence identity may be adjusted upwards to account for the similarity in conservatively substituted amino acids. Such adjustments are well-known in the art (see, e.g., Meyers and Miller (1988) Computer Applic. Bio. Sci., 4: 11-17).

To prepare an active mutein of an FGF2 of the present invention, one can use standard techniques for site directed mutagenesis, as known in the art and/or as taught in Gilman et al. (1979) Gene, 8:81 or Roberts et al. (1987) Nature, 328: 731. Using one of the site directed mutagenesis techniques, one or more point mutations are introduced into the cDNA sequence of encoding the FGF2 or FGF2 fragment to introduce one or more amino acid substitutions or an internal deletion. Conservative amino acid substitutions are those that preserve the general charge, hydrophobicity/hydrophilicity, and/or steric bulk of the amino acid being substituted. By way of example, substitutions between the following groups are conservative: Gly/Ala, Val/Ile/Leu, Lys/Arg, Asn/Gln, Glu/Asp, Ser/Cys/Thr, and Phe/Trp/Tyr. Significant (up to 35%) variation from the sequence of the naturally occurring angiogenic FGF2 is permitted as long as the resulting protein or polypeptide retains activity within the limits specified above.

In certain embodiments, the FGF2 muteins include, but are not limited to cysteine-depleted muteins. A cysteine depleted mutein is a mutein in which one or more of the cysteines in the naturally occurring FGF2 are replaced with a different amino acid (e.g. a serine). Cysteine-depleted muteins can be constructed using site directed mutagenesis as described above, or according to the method described in U.S. Pat. No. 4,959,314, which is incorporated herein by reference. This patent discloses how to determine biological activity and the effect of the substitution. Cysteine substitution is particularly useful in proteins having two or more cysteines that are not involved in disulfide formation. Suitable substitutions include the substitution of serine for one or both of the cysteines at residue positions 87 and 92, which are not involved in disulfide formation. Preferably, substitutions are introduced at the FGF2 N-terminus, which is not associated with angiogenic activity. However, as discussed above, conservative substitutions are suitable for introduction throughout the molecule.

The muteins described above are intended to be illustrative and not limiting. In general, other suitable muteins can readily be identified by introducing one or more mutations into FGF2 and screening the resulting muteins for the desired biological activity (e.g., angiogenic activity) using methods well known to those of skill in the art.

### II. Pharmaceutical Formulations.

In certain embodiments, in order to carry out the methods of the invention, active agent(s) (e.g., FGF2, muteins, mimetics, or analogues thereof, vectors encoding FGF2, drugs that upregulate FGF2, and the like) of this invention are administered, e.g. to an individual diagnosed as having a neurodegenerative pathology (e.g., Huntington's disease, Parkinson's disease, and the like), or as being at risk for a neurodegenerative pathology. The active agent(s) can be administered in the “native” form or, if desired, in the form of salts, esters, amides, prodrugs, derivatives, and the like, provided the salt, ester, amide, prodrug or derivative is suitable pharmacologically, i.e., effective in the present method. Salts, esters, amides, prodrugs and other derivatives of the active agents can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

For example, acid addition salts are prepared from the free base using conventional methodology, that typically involves reaction with a suitable acid. Generally, the base form of the drug is dissolved in a polar organic solvent such as methanol or ethanol and the acid is added thereto. The resulting salt either precipitates or can be brought out of solution by addition of a less polar solvent. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Particularly preferred acid addition salts of the active agents herein are halide salts, such as may be prepared using hydrochloric or hydrobromic acids. Conversely, preparation of basic salts of the active agents of this invention are prepared in a similar manner using a pharmaceutically acceptable base such as sodium hydroxide, potassium hydroxide, ammonium hydroxide, calcium hydroxide, trimethylamine, or the like. Particularly preferred basic salts include alkali metal salts, e.g., the sodium salt, and copper salts.

Preparation of esters typically involves functionalization of hydroxyl and/or carboxyl groups which may be present within the molecular structure of the drug. The esters are typically acyl-substituted derivatives of free alcohol groups, i.e., moieties that are derived from carboxylic acids of the formula RCOOH where R is alky, and preferably is lower alkyl. Esters can be reconverted to the free acids, if desired, by using conventional hydrogenolysis or hydrolysis procedures.

Amides and prodrugs can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine. Prodrugs are typically prepared by covalent attachment of a moiety that results in a compound that is therapeutically inactive until modified by an individual's metabolic system.

The active agents identified herein (e.g. FGF2, FGF2 muteins, etc.) are useful for parenteral, topical, oral, nasal (or otherwise inhaled), rectal, or local administration, such as by aerosol or transdermally, for prophylactic and/or therapeutic treatment of one or more of the pathologies/indications described herein (e.g., atherosclerosis and/or symptoms thereof). The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectibles, implantable sustained-release formulations, lipid complexes, etc.

The active agents of this invention are typically combined with a pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, protection and uptake enhancers such as lipids, compositions that reduce the clearance or hydrolysis of the active agents, or excipients or other stabilizers and/or buffers.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s).

The excipients are preferably sterile and generally free of undesirable matter. These compositions may be sterilized by conventional, well-known sterilization techniques.

In therapeutic applications, the compositions of this invention are administered to a patient suffering from one or more symptoms of the one or more pathologies described herein, e.g., Huntington's disease, or at risk for one or more of the pathologies described herein (e.g. Huntington's disease) in an amount sufficient to prevent and/or cure and/or or at least partially prevent or arrest the disease and/or its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the active agents of the formulations of this invention to effectively treat (ameliorate one or more symptoms) the patient.

The concentration of active agent(s) can vary widely, and will be selected primarily based on fluid volumes, viscosities, body weight and the like in accordance with the particular mode of administration selected and the patient's needs. Concentrations, however, will typically be selected to provide dosages ranging from about 0.1 or 1 mg/kg/day to about 50 mg/kg/day and sometimes higher. In certain embodiments typical dosages range from about 3 mg/kg/day to about 3.5 mg/kg/day, preferably from about 3.5 mg/kg/day to about 7.2 mg/kg/day, more preferably from about 7.2 mg/kg/day to about 11.0 mg/kg/day, and most preferably from about 11.0 mg/kg/day to about 15.0 mg/kg/day. In certain preferred embodiments, dosages range from about 10 mg/kg/day to about 50 mg/kg/day. In certain embodiments, dosages range from about 20 mg to about 50 mg given orally twice daily.

In certain embodiments the safe and effective dose of the pharmaceutical composition of the present invention in a form and a size suitable for administration to a human patient and comprises (i) 0.2 μg/kg to 48 μg/kg of FGF2 or FGF2 mutein or fragment. In other embodiments, the safe and angiogenically effective dose comprises 0.2 μg/kg to 2 μg/kg, >2.4 g/kg to <24 μg/kg or 24 μg/kg to 48 μg/kg of FGF2, FGF2 mutein or fragment. Expressed in absolute terms, in certain embodiments the unit dose of the present invention comprises 0.008 mg to 7.2 mg, more typically 0.3 mg to 3.5 mg, of the FGF2, FGF2 mutein or fragment.

It will be appreciated that such dosages may be varied to optimize a therapeutic regimen in a particular subject or group of subjects.

In certain preferred embodiments, the active agents of this invention are administered orally (e.g. via a tablet) or as an injectable in accordance with standard methods well known to those of skill in the art. In other preferred embodiments, the peptides, may also be delivered through the skin using conventional transdermal drug delivery systems, i.e., transdermal “patches” wherein the active agent(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the active agent(s) and any other materials that are present.

Other preferred formulations for topical drug delivery include, but are not limited to, ointments and creams. Ointments are semisolid preparations which are typically based on petrolatum or other petroleum derivatives. Creams containing the selected active agent, are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. The specific ointment or cream base to be used, as will be appreciated by those skilled in the art, is one that will provide for optimum drug delivery. As with other carriers or vehicles, an ointment base should be inert, stable, nonirritating and nonsensitizing.

In certain embodiments, peptide (e.g. FGF2) delivery can be enhanced by the use of protective excipients. This is typically accomplished either by complexing the polypeptide with a composition to render it resistant to acidic and enzymatic hydrolysis or by packaging the polypeptide in an appropriately resistant carrier such as a liposome. Means of protecting polypeptides for oral delivery are well known in the art (see, e.g., U.S. Pat. No. 5,391,377 describing lipid compositions for oral delivery of therapeutic agents).

Elevated serum half-life can be maintained by the use of sustained-release protein “packaging” systems. Such sustained release systems are well known to those of skill in the art. In one preferred embodiment, the ProLease biodegradable microsphere delivery system for proteins and peptides (Tracy (1998) Biotechnol. Prog. 14: 108; Johnson et al. (1996), Nature Med. 2: 795; Herbert et al. (1998), Pharmaceut. Res. 15, 357) a dry powder composed of biodegradable polymeric microspheres containing the active agent in a polymer matrix that can be compounded as a dry formulation with or without other agents.

The ProLease microsphere fabrication process was specifically designed to achieve a high encapsulation efficiency while maintaining integrity of the active agent. The process consists of (i) preparation of freeze-dried drug particles from bulk by spray freeze-drying the drug solution with stabilizing excipients, (ii) preparation of a drug-polymer suspension followed by sonication or homogenization to reduce the drug particle size, (iii) production of frozen drug-polymer microspheres by atomization into liquid nitrogen, (iv) extraction of the polymer solvent with ethanol, and (v) filtration and vacuum drying to produce the final dry-powder product. The resulting powder contains the solid form of the active agents, which is homogeneously and rigidly dispersed within porous polymer particles. The polymer most commonly used in the process, poly(lactide-co-glycolide) (PLG), is both biocompatible and biodegradable.

Encapsulation can be achieved at low temperatures (e.g., −40° C.). During encapsulation, the protein is maintained in the solid state in the absence of water, thus minimizing water-induced conformational mobility of the protein, preventing protein degradation reactions that include water as a reactant, and avoiding organic-aqueous interfaces where proteins may undergo denaturation. A preferred process uses solvents in which most proteins are insoluble, thus yielding high encapsulation efficiencies (e.g., greater than 95%).

In another embodiment, one or more components of the solution can be provided as a “concentrate”, e.g., in a storage container (e.g., in a premeasured volume) ready for dilution, or in a soluble capsule ready for addition to a volume of water.

FGF2 and certain FGF2 muteins can lose activity in aqueous solutions. In various embodiments inactivation can be prevented by the addition of the glycosaminoglycan, heparin (see, e.g., (1986) J. Cell. Physiol., 128: 475). In certain embodiments, the FGF2 or FGF2 mutein is stabilized by formulation with a glucan sulfate. Methods of formulating FGF2 and FGF2 muteins with a glucan sulfate are described in U.S. Pat. No. 5,314,872, which is incorporated herein by reference.

The foregoing formulations and administration methods are intended to be illustrative and not limiting. It will be appreciated that, using the teaching provided herein, other suitable formulations and modes of administration can be readily devised.

III. Transfection of a Host with Vectors Encoding FGF2 or FGF2 Muteins.

In certain embodiments, the FGF2 or FGF2 mutein is delivered to the subject by transducing/transforming the subject with an expression vector encoding the FGF2 or FGF2 mutein, e.g., as described herein, operably linked to a constitutive, tissue specific, or inducible promoter. Such expression vectors include, but are not limited to, eukaryotic vectors, prokaryotic vectors (such as, for example, bacterial vectors), and viral vectors. In one alternative embodiment, the polynucleotide encoding the FGF2 or FGF2 mutein, or an expression vehicle containing the polynucleotide encoding the FGF2 or FGF2 mutein, is contained within a liposome.

Many approaches for introducing nucleic acids into cells in vivo, ex vivo and in vitro are known to those of skill in the art. These include, but are not limited to lipid or liposome based gene delivery (see, e.g., WO 96/18372; WO 93/24640; Mannino and Gould-Fogerite (1988) BioTechniques 6(7): 682-691; Rose U.S. Pat. No. 5,279,833; WO 91/06309; and Felgner et al. (1987) Proc. Natl. Acad. Sci. USA 84: 7413-7414), electroporation, calcium phosphate transfection, viral vectors, biolistics, microinjection, dendrimer conjugation, and the like. In particularly preferred embodiments, transfection is by means of replication-defective retroviral vectors (see, e.g., Miller et al. (1990) Mol. Cell. Biol. 10:4239 (1990); Kolberg (1992) J. NIH Res. 4: 43, and Cometta et al. (1991) Hum. Gene Ther. 2: 215).

For a review of gene therapy procedures, see, e.g., Anderson (1992) Science 256: 808-813; Nabel and Felglier (1993) TIBTECH 11: 211-217; Mitani and Caskey (1993) TIBTECH 11: 162-166; Mulligan (1993) Science, 926-932; Dillon (1993) TIBTECH 11: 167-175; Miller (1992) Nature 357: 455-460; Van Brunt (1988) Biotechnology 6(10): 1149-1154; Vigne (1995) Restorative Neurology and Neuroscience 8: 35-36; Kremer and Perricaudet (1995) British Medical Bulletin 51(1) 31-44; Haddada et al. (1995) in Current Topics in Microbiology and Immunology, Doerfler and Bohm (eds) Springer-Verlag, Heidelberg Germany; and Yu et al., (1994) Gene Therapy, 1:13-26.

Widely used vectors include those based upon murine leukemia virus (MuLV), gibbon ape leukemia virus (GaLV), Simian Immunodeficiency virus (SIV), human immunodeficiency virus (HIV), alphavirus, and combinations thereof (see, e.g., Buchscher et al. (1992) J. Virol. 66(5) 2731-2739; Johann et al. (1992) J. Virol. 66 (5):1635-1640 (1992); Sommerfelt et al., (1990) Virol. 176:58-59; Wilson et al. (1989) J. Virol. 63:2374-2378; Miller et al., J. Virol. 65:2220-2224 (1991); Wong-Staal et al., PCT/US94/05700, and Rosenburg and Fauci (1993) in Fundamental Immunology, Third Edition Paul (ed) Raven Press, Ltd., New York and the references therein, and Yu et al. (1994) Gene Therapy, supra; U.S. Pat. No. 6,008,535, and the like).

The construction and use of various gene therapy vectors is also described in U.S. Pat. No. 7,074,772, U.S. Pat. No. 7,064,111, U.S. Pat. No. 7,052,881, U.S. Pat. No. 7,037,716, RE39,078, U.S. Pat. No. 7,022,319, U.S. Pat. No. 7,018,826, U.S. Pat. No. 7,001,760, and the like which are incorporated herein by reference.

The vectors are optionally pseudotyped to extend the host range of the vector to cells which are not infected by the retrovirus corresponding to the vector. For example, the vesicular stomatitis virus envelope glycoprotein (VSV-G) has been used to construct VSV-G-pseudotyped HIV vectors which can infect hematopoietic stem cells (Naldini et al. (1996) Science 272:263, and Akkina et al. (1996) J Virol 70:2581).

Adeno-associated virus (AAV)-based vectors are also used to transduce cells with target nucleic acids, e.g., in the in vitro production of nucleic acids and peptides, and in in vivo and ex vivo gene therapy procedures. See, West et al. (1987) Virology 160:38-47; Carter et al. (1989) U.S. Pat. No. 4,797,368; Carter et al. WO 93/24641 (1993); Kotin (1994) Human Gene Therapy 5:793-801; Muzyczka (1994) J. Clin. Invst. 94:1351 for an overview of AAV vectors. Construction of recombinant AAV vectors are described in a number of publications, including Lebkowski, U.S. Pat. No. 5,173,414; Tratschin et al. (1985) Mol. Cell. Biol. 5(11):3251-3260; Tratschin, et al. (1984) Mol. Cell. Biol., 4: 2072-2081; Hermonat and Muzyczka (1984) Proc. Natl. Acad. Sci. USA, 81: 6466-6470; McLaughlin et al. (1988) and Samulski et al. (1989) J. Virol., 63:03822-3828. Cell lines that can be transformed by rAAV include those described in Lebkowski et al. (1988) Mol. Cell. Biol., 8:3988-3996. Other suitable viral vectors include herpes virus, lentivirus, and vaccinia virus.

In one particularly preferred embodiment, retroviruses (e.g. lentiviruses) are used to transfect the target cell(s) with nucleic acids encoding the FGF2 or FGF2 mutein. Retroviruses, in particular lentiviruses (e.g. HIV, SIV, etc.) are particularly well suited for this application because they are capable of infecting a non-dividing cell. Methods of using retroviruses for nucleic acid transfection are known to those of skill in the art (see, e.g., U.S. Pat. No. 6,013,576).

Retroviruses are RNA viruses wherein the viral genome is RNA. When a host cell is infected with a retrovirus, the genomic RNA is reverse transcribed into a DNA intermediate which is integrated very efficiently into the chromosomal DNA of infected cells. This integrated DNA intermediate is referred to as a provirus. Transcription of the provirus and assembly into infectious virus occurs in the presence of an appropriate helper virus or in a cell line containing appropriate sequences enabling encapsidation without coincident production of a contaminating helper virus. In preferred embodiments, a helper virus need not be utilized for the production of the recombinant retrovirus since the sequences for encapsidation can be provided by co-transfection with appropriate vectors. The retroviral genome and the proviral DNA have three genes: the gag, the pol, and the env, which are flanked by two long terminal repeat (LTR) sequences. The gag gene encodes the internal structural (matrix, capsid, and nucleocapsid) proteins; the pol gene encodes the RNA-directed DNA polymerase (reverse transcriptase) and the env gene encodes viral envelope glycoproteins. The 5′ and 3′ LTRs serve to promote transcription and polyadenylation of the virion RNAs. The LTR contains all other cis-acting sequences necessary for viral replication. Lentiviruses have additional genes including vit vpr, tat, rev, vpu, nef, and vpx (in HIV-1, HIV-2 and/or SIV).

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the result is a cis defect which prevents encapsidation of genomic RNA. However, the resulting mutant is still capable of directing the synthesis of all virion proteins.

In one preferred embodiment, the invention provides a recombinant retrovirus capable of infecting a non-dividing cell. The recombinant retrovirus comprises a viral GAG, a viral POL, a viral ENV, a heterologous nucleic acid sequence operably linked to a regulatory nucleic acid sequence, and cis-acting nucleic acid sequences necessary for packaging, reverse transcription and integration, as described above. It should be understood that the recombinant retrovirus of the invention is capable of infecting dividing cells as well as non-dividing cells.

In preferred embodiments, the recombinant retrovirus is therefore genetically modified in such a way that some of the structural, infectious genes of the native virus (e.g. env, gag, pol have been removed and replaced instead with a nucleic acid sequence to be delivered to a target non-dividing cell (e.g., a sequence encoding the reporter and/or cytotoxic gene under control of the HPV promoter). After infection of a cell by the virus, the virus injects its nucleic acid into the cell and the retrovirus genetic material can, optionally, integrate into the host cell genome. Methods of making and using lentiviral vectors are discussed in detail in U.S. Pat. Nos. 6,013,516, 5,932,467, and the like.

It is also noted that the construction of and intracerebroventricular administration of an FGF2-expressing herpes simplex virus amplicon vector is described by Yoshimura et al. (2001) Proc. Natl. Acad. Sci., USA, 98:5874-5879.

In another embodiment, the nucleic acid encoding the FGF2 and/or FGF2 mutein(s) are placed in an adenoviral vector suitable for gene therapy. The use of adenoviral vectors is described in detail in WO 96/25507. Particularly preferred adenoviral vectors are described by Wills et al. (1994) Hum. Gene Therap. 5: 1079-1088. Typically, adenoviral vectors contain a deletion in the adenovirus early region 3 and/or early region 4 and this deletion may include a deletion of some, or all, of the protein IX gene. In one embodiment, the adenoviral vectors include deletions of the E1a and/or E1b sequences.

A number of different adenoviral vectors have been optimized for gene transfer. One such adenoviral vector is described in U.S. Pat. No. 6,020,191. This vector comprises a CMV promoter to which a transgene may be operably linked and further contains an E1 deletion and a partial deletion of 1.6 kb from the E3 region. This is a replication defective vector containing a deletion in the E1 region into which a transgene (e.g. the β subunit gene) and its expression control sequences can be inserted, preferably the CMV promoter contained in this vector. It further contains the wild-type adenovirus E2 and E4 regions. The vector contains a deletion in the E3 region which encompasses 1549 nucleotides from adenovirus nucleotides 29292 to 30840 (Roberts et al. (1986) Adenovirus DNA, in Developments in Molecular Virology, W. Doerfler, ed., 8: 1-51). These modifications to the E3 region in the vector result in the following: (a) all the downstream splice acceptor sites in the E3 region are deleted and only mRNA a would be synthesized from the E3 promoter (Tollefson et al. (1996) J, Virol. 70:2 296-2306, 1996; Tollefson et al. (1996) Virology 220: 152-162,); (b) the E3A poly A site has been deleted, but the E3B poly A site has been retained; (c) the E3 gp19K (MHC I binding protein) gene has been retained; and (d) the E3 11.6K (Ad death protein) gene has been deleted.

Such adenoviral vectors can utilize adenovirus genomic sequences from any adenovirus serotypes, including but not limited to, adenovirus serotypes 2, 5, and all other preferably non-oncogenic serotypes.

Alone, or in combination with viral vectors, a number of non-viral vectors are also useful for transfecting cells with reporter and/or cytotoxic genes under control of the HPV promoter. Suitable non-viral vectors include, but are not limited to, plasmids, cosmids, phagemids, liposomes, water-oil emulsions, polethylene imines, biolistic pellets/beads, and dendrimers.

Cationic liposomes are positively charged liposomes that interact with the negatively charged DNA molecules to form a stable complex. Cationic liposomes typically consist of a positively charged lipid and a co-lipid. Commonly used co-lipids include dioleoyl phosphatidylethanolamine (DOPE) or dioleoyl phosphatidylcholine (DOPC). Co-lipids, also called helper lipids, are in most cases required for stabilization of liposome complex. A variety of positively charged lipid formulations are commercially available and many others are under development. Two of the most frequently cited cationic lipids are lipofectamine and lipofectin. Lipofectin is a commercially available cationic lipid first reported by Phil Felgner in 1987 to deliver genes to cells in culture. Lipofectin is a mixture of N-[1-(2,3-dioleyloyx) propyl]-N—N—N-trimethyl ammonia chloride (DOTMA) and DOPE.

DNA and lipofectin or lipofectamine interact spontaneously to form complexes that have a 100% loading efficiency. In other words, essentially all of the DNA is complexed with the lipid, provided enough lipid is available. It is assumed that the negative charge of the DNA molecule interacts with the positively charged groups of the DOTMA. The lipid:DNA ratio and overall lipid concentrations used in forming these complexes are extremely important for efficient gene transfer and vary with application. Lipofectin has been used to deliver linear DNA, plasmid DNA, and RNA to a variety of cells in culture. Shortly after its introduction, it was shown that lipofectin could be used to deliver genes in vivo. Following intravenous administration of lipofectin-DNA complexes, both the lung and liver showed marked affinity for uptake of these complexes and transgene expression. Injection of these complexes into other tissues has had varying results and, for the most part, are much less efficient than lipofectin-mediated gene transfer into either the lung or the liver.

PH-sensitive, or negatively-charged liposomes, entrap DNA rather than complex with it. Since both the DNA and the lipid are similarly charged, repulsion rather than complex formation occurs. Yet, some DNA does manage to get entrapped within the aqueous interior of these liposomes. In some cases, these liposomes are destabilized by low pH and hence the term pH-sensitive. To date, cationic liposomes have been much more efficient at gene delivery both in vivo and in vitro than pH-sensitive liposomes. pH-sensitive liposomes have the potential to be much more efficient at in vivo DNA delivery than their cationic counterparts and should be able to do so with reduced toxicity and interference from serum protein.

In another approach dendrimers complexed to the DNA have been used to transfect cells. Such dendrimers include, but are not limited to, “starburst” dendrimers and various dendrimer polycations.

Dendrimer polycations are three dimensional, highly ordered oligomeric and/or polymeric compounds typically formed on a core molecule or designated initiator by reiterative reaction sequences adding the oligomers and/or polymers and providing an outer surface that is positively changed. These dendrimers may be prepared as disclosed in PCT/US83/02052, and U.S. Pat. Nos. 4,507,466, 4,558,120, 4,568,737, 4,587,329, 4,631,337, 4,694,064, 4,713,975, 4,737,550, 4,871,779, 4,857,599.

Typically, the dendrimer polycations comprise a core molecule upon which polymers are added. The polymers may be oligomers or polymers which comprise terminal groups capable of acquiring a positive charge. Suitable core molecules comprise at least two reactive residues which can be utilized for the binding of the core molecule to the oligomers and/or polymers. Examples of the reactive residues are hydroxyl, ester, amino, imino, imido, halide, carboxyl, carboxyhalide maleimide, dithiopyridyl, and sulfhydryl, among others. Preferred core molecules are ammonia, tris-(2-aminoethyl)amine, lysine, ornithine, pentaerythritol and ethylenediamine, among others. Combinations of these residues are also suitable as are other reactive residues.

Oligomers and polymers suitable for the preparation of the dendrimer polycations of the invention are pharmaceutically-acceptable oligomers and/or polymers that are well accepted in the body. Examples of these are polyamidoamines derived from the reaction of an alkyl ester of an α,β-ethylenically unsaturated carboxylic acid or an α,β-ethylenically unsaturated amide and an alkylene polyamine or a polyalkylene polyamine, among others. Preferred are methyl acrylate and ethylenediamine. The polymer is preferably covalently bound to the core molecule.

The terminal groups that may be attached to the oligomers and/or polymers should be capable of acquiring a positive charge. Examples of these are azoles and primary, secondary, tertiary and quaternary aliphatic and aromatic amines and azoles, which may be substituted with S or O, guanidinium, and combinations thereof. The terminal cationic groups are preferably attached in a covalent manner to the oligomers and/or polymers. Preferred terminal cationic groups are amines and guanidinium. However, others may also be utilized. The terminal cationic groups may be present in a proportion of about 10 to 100% of all terminal groups of the oligomer and/or polymer, and more preferably about 50 to 100%.

The dendrimer polycation may also comprise 0 to about 90% terminal reactive residues other than the cationic groups. Suitable terminal reactive residues other than the terminal cationic groups are hydroxyl, cyano, carboxyl, sulfhydryl, amide and thioether, among others, and combinations thereof. However others may also be utilized.

The dendrimer polycation is generally and preferably non-covalently associated with the polynucleotide. This permits an easy disassociation or disassembling of the composition once it is delivered into the cell. Typical dendrimer polycations suitable for use herein have a molecular weight ranging from about 2,000 to 1,000,000 Da, and more preferably about 5,000 to 500,000 Da. However, other molecule weights are also suitable. Preferred dendrimer polycations have a hydrodynamic radius of about 11 to 60 Å., and more preferably about 15 to 55 Å. Other sizes, however, are also suitable. Methods for the preparation and use of dendrimers in gene therapy are well known to those of skill in the art and describe in detail, for example, in U.S. Pat. No. 5,661,025.

Where appropriate, two or more types of vectors can be used together. For example, a plasmid vector may be used in conjunction with liposomes. In the case of non-viral vectors, nucleic acid may be incorporated into the non-viral vectors by any suitable means known in the art. For plasmids, this typically involves ligating the construct into a suitable restriction site. For vectors such as liposomes, water-oil emulsions, polyethylene amines and dendrimers, the vector and construct may be associated by mixing under suitable conditions known in the art.

Vectors (e.g., retroviruses, adenoviruses, liposomes, etc.) containing therapeutic nucleic acids can be administered directly to the organism for transduction of cells in vivo. Administration is by any of the routes normally used for introducing a molecule into ultimate contact with blood or tissue cells. The nucleic acids are administered in any suitable manner, preferably with pharmaceutically acceptable carriers. Suitable methods of administering such packaged nucleic acids are available and well known to those of skill in the art.

### IV. Upregulation of Endogenous FGF2.

In certain embodiments subjects having or at risk for a neurodegenerative disease can be effectively administered endogenous FGF2 by upregulating expression of the endogenous molecule. In certain embodiments FGF2 expression can be upregulated by modification of the endogenous FGF2 promoter. Methods of modifying or replacing native promoters to alter expression of endogenous genes are well known to those of skill in the art (see, e.g., U.S. Pat. Nos. 5,272,071, WO 91/09955, WO 93/09222, WO 96/29411, WO 95/31560, and WO 91/12650).

Alternatively, the subjects can be administered agents that upregulate endogenous FGF2 levels. In this context, it is noted, for example, that particle radiations, including both proton and helium-ion beams, have been used to successfully treat choroidal melanoma, with the accompanying “complication” of radiation-induced changes in the expression of basic fibroblast growth factor (FGF2) gene expression as part of the mechanism(s) underlying lens cell injury associated with cataract formation (see, e.g., Chang et al. ( ) Radiation Res., 154(5): 477-484).

A number of drugs have also been shown to increase FGF2 levels. Thus, for example, the use of antidepressants (e.g., desipramine (DMI), fluoxetine (FLU), and mianserin (MIA)) has been shown to increase FGF2 levels. In particular, for example, DMI and MIA increased FGF2 proteins predominantly in neurons of layer V throughout the cerebral cortex and in some neurofilament-positive cells of the hippocampus, while FLU increased FGF2 immunoreactivity mainly in neurofilament-positive cells of the hippocampus (see, e.g., Mallei et al. (2002) Molecular Pharmacology., 61(5): 1017-1024).

Activation of the central noradrenergic system, as obtained by activation of β2-adrenergic receptors (Follesa and Mocchetti (1993) Mol Pharmacol 43: 132-138; Hayes et al. (1995) Exp Neurol 132: 33-41) or experimental electroshock (Follesa et al. (1994) Exp Neurol 127: 37-44; Gall et al. (1994) Mol Brain Res 21: 190-205) has also been shown to increase the synthesis of basic fibroblast growth factor (FGF2) in selected areas of the brain. Thus, agents that activate β2-adrenergic receptors can also be used to upregulate FGF2. Beta 2-adrenergic receptor agonists are known to those of skill in the art (see, e.g., formoterol).

Other agents that upregulate FGF2 expression can readily be identified by administering the agent(s) in question to a test animal and assaying the test animal for increased FGF2 expression by methods well known to those of skill in the art.

### V. Kits.

In another embodiment this invention provides kits for amelioration of one or more symptoms of a pathology characterized by neurodegeneratino (e.g. Parkinson's disease, Huntington's disease, etc.) and/or for the prophylactic treatment of a subject (human or animal) at risk for a neurodegenerative pathology. The kits preferably comprise a container containing one or more of the FGF2 molecules, FGF2 muteins, or various other therapeutic agents as described herein. The FGF2 or FGF2 mutein or mimetic can be provided in a unit dosage formulation (e.g. suppository, tablet, caplet, patch, etc.) and/or may be optionally combined with one or more pharmaceutically acceptable excipients.

In addition, the kits optionally include labeling and/or instructional materials providing directions (i.e., protocols) for the practice of the methods or use of the “therapeutics” or “prophylactics” of this invention. Preferred instructional materials describe the use of one or more agents of this invention to mitigate one or more symptoms of a neurodegenerative pathology and/or to prevent the onset or increase of one or more of such symptoms in an individual at risk for a neurodegenerative pathology. The instructional materials may also, optionally, teach preferred dosages/therapeutic regiment, counter indications and the like.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Fibroblast Growth Factor-2 Promotes Neurogenesis and Neuroprotection and Prolongs Survival in a Transgenic Mouse Model of Huntington's Disease

There is no satisfactory treatment for Huntington's disease (HD), a hereditary neurodegenerative disorder that produces chorea, dementia and death. One potential treatment strategy involves the replacement of dead neurons by stimulating the proliferation of endogenous neuronal precursors (neurogenesis) and their migration into damaged regions of the brain. Because growth factors are neuroprotective in some settings and can also stimulate neurogenesis, we treated HD transgenic R6/2 mice from eight weeks of age until death by subcutaneous administration of fibroblast growth factor-2 (FGF-2). FGF-2 increased the number of proliferating cells in the subventricular zone (SVZ) by ˜30% in wild-type mice, and by ˜150% in HD transgenic R6/2 mice. FGF-2 also induced the recruitment of new neurons from the SVZ into the neostriatum and cerebral cortex of HD transgenic R6/2 mice. In the striatum, these new neurons were DARPP-32-expressing medium spiny neurons, consistent with the phenotype of neurons lost in HD. FGF-2 was neuroprotective as well, since it blocked cell death induced by mutant expanded Htt in primary striatal cultures. FGF-2 also reduced polyglutamine aggregates, improved motor performance, and extended lifespan by ˜20%. We conclude that FGF-2 improves neurological deficits and longevity in a transgenic mouse model of HD, and that its neuroprotective and neuroproliferative effects may contribute to this improvement.

### Results

Basal and FGF-2-Stimulated Neurogenesis is Increased in SVZ of HD Transgenic R6/2 Mice

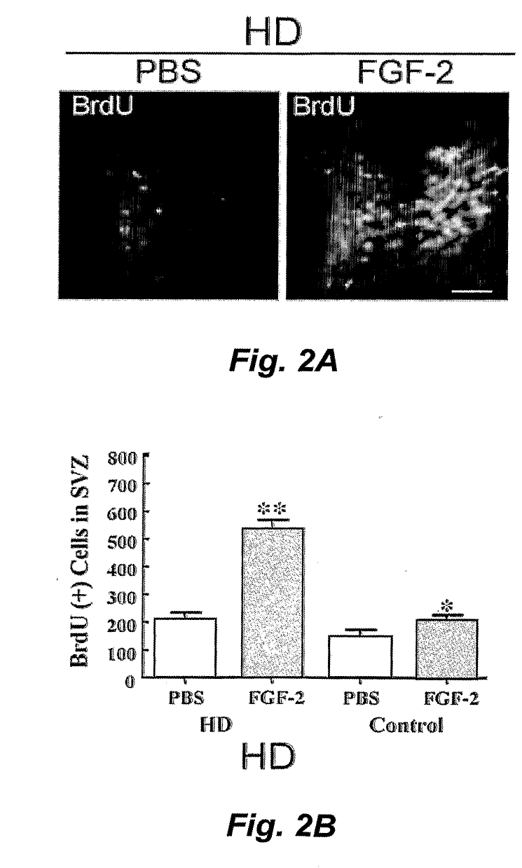

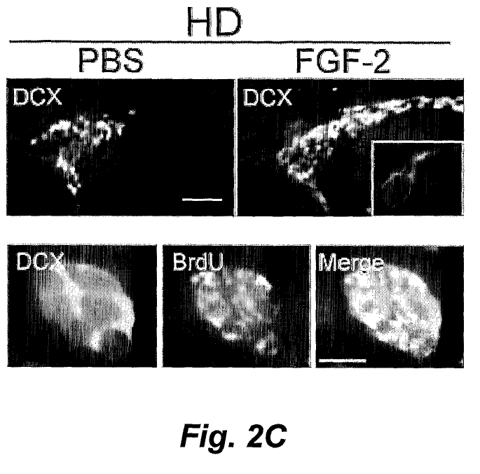

Growth factors can stimulate neurogenesis in some settings, but whether they can do so in genetic HD mouse models is not known. Compared to age-matched controls, brain from HD patients expresses increased levels of PCNA, a mitotic marker protein (Curtis et al. (2003) Proc Natl Acad Sci USA 100: 9023-9027), and Htt itself may be required for neurogenesis (White et al. (1997) Nat Genet 17: 404-410). To measure basal and FGF-2-stimulated neurogenesis, vehicle or FGF-2 was injected subcutaneously in 8-week old HD transgenic R6/2 mice and wild-type littermate controls for three weeks. Bromodeoxyuridine (BrdU), used to detect proliferating cells, was injected intraperitoneally for 3 days, and animals were killed 24 hour later for BrdU immunohistochemistry (FIG. 2a). Neurogenesis was measured by counting BrdU-labeled cells in the two principal neuroproliferative regions of the adult rodent brain: the SVZ and the subgranular zone (SGZ) of the hippocampal dentate gyrus (DG). Like HD patients, untreated HD transgenic R6/2 mice showed a statistically significant increase in the number of proliferating cells in the SVZ compared to controls, but the magnitude of increase was modest (FIG. 2b, *p<0.05). However, FGF-2 increased BrdU labeling in SVZ of HD transgenic R6/2 mice by ˜150%, while in control mice the magnitude of increase was only ˜30% (FIGS. 2a,b). In contrast to SVZ, there was no increase in BrdU labeling in DG (not shown) upon FGF-2 treatment in control and HD transgenic R6/2 mice. Untreated 8-week old HD transgenic R6/2 mice and wild-type littermate controls had similar BrdU labeling in the DG as has been previously reported (Lazic et al. (2004) Neuroreport 15: 811-813).

Staining for doublecortin, a marker for newborn and migrating neurons, was also increased in the SVZ of the HD transgenic R6/2 mice (198%+/−20% compared to control), suggesting migration of these cells from the SVZ into affected areas of the brain ( ) in the presence of FGF-2. As previously observed in young adult mice (Jin et al. (2003) Aging Cell 2: 175-183), FGF-2 did not increase proliferating cells in the hippocampus (data not shown). These results demonstrate that neurogenesis is increased in SVZ of HD transgenic R6/2 mice, and that FGF-2 stimulates neurogenesis to a greater extent in these mice than in controls.

New neurons in FGF-2-Treated HD Transgenic R6/2 Mice Express Phenotypic Features of Medium Spiny Neurons

Virally-induced expression of brain-derived neurotrophic factor (BDNF) together with noggin, which inhibits glial differentiation, can stimulate the production of striatal neurons expressing markers of medium spiny neurons (the principal cell type lost in HD), including calbindin, glutamic acid decarboxylase (GAD67), and DARPP-32, a dopamine-regulated phosphoprotein (Chmielnicki et al. (2004) J Neurosci 24: 2133-2142). To determine if FGF-2 has a similar effect, we double-labeled brain sections taken from HD transgenic R6/2 mice, treated with FGF-2 and killed at 11 weeks, with antibodies against DCX and DARPP-32 (FIG. 3a). Cells migrating into the striatum expressed DARPP-32 (FIG. 3a), suggesting maturation along a lineage appropriate for medium spiny neurons. In cerebral cortex, some DCX-expressing cells also expressed the neuronal marker NeuN, consistent with continuing differentiation towards a mature neuronal phenotype (FIG. 3a).

Newly Generated Neurons Develop Projections to the Globus Pallidus

Next, we tested whether the new neurons in HD transgenic R6/2 mice extend processes to their normal target, the globus pallidus. To address this question, we injected the retrograde tracer Alexa Fluoro 488 into the globus pallidus of HD transgenic R6/2 mice treated with FGF-2 (FIG. 3b, panel a) and given BrdU. One week after FGF-2 treatment and 72 hours after injection of the tracer, the mice were killed, and striata were examined for the presence of DCX, NeuN, Alexa Fluoro 488 and BrdU. The coexpression of DCX, NeuN and Alexa Fluoro 488 (FIG. 3b), and of BrdU with Alexa Fluoro 488 (FIG. 3c, left panel), indicates that newly produced striatal neurons form projections to globus pallidus. DARPP-32, a marker of mature striatal neurons, was also expressed in BrdU labeled striatal cells (FIG. 3c, right panel). FGF-2 treatment appears to be responsible for the presence of newborn neurons in the striatum of HD transgenic R6/2 mice, because no DCX/BrdU immunopositive striatal cells were found in mice given PBS (data not shown).

To evaluate the functional consequences of FGF-2 treatment, we measured lifespan and motor function in HD transgenic R6/2 mice, as described (Hockly et al. (2003) Brain Res Bull 61: 469-479). FGF-2 or PBS was administered by subcutaneous injection twice daily for 3 days per week, starting at 59 days of age. The rotarod latency and weight of FGF-2 treated and untreated mice were the same prior to treatment and each treatment group had the same composition of females and males. FGF-2 delayed the onset of mortality in HD transgenic R6/2 mice from 84 to 100 days, the average survival from 102 to 123 days, and the maximum survival from 118 to 145 days (p<0.05). FGF-2 also reduced tremor (data not shown) and improved rotarod performance measured at 11 and 13 weeks of age (, p<0.01). Finally, FGF-2 treatment produced a modest decrement in weight loss in HD transgenic R6/2 mice, which reached statistical significance at 13 weeks of age, when mean weights were 17.6±1.2 g for FGF-2-treated and 15.4±0.8 g for PBS-treated mice (p<0.05).

FGF-2 is Neuroprotective in Cell Culture Models of HD

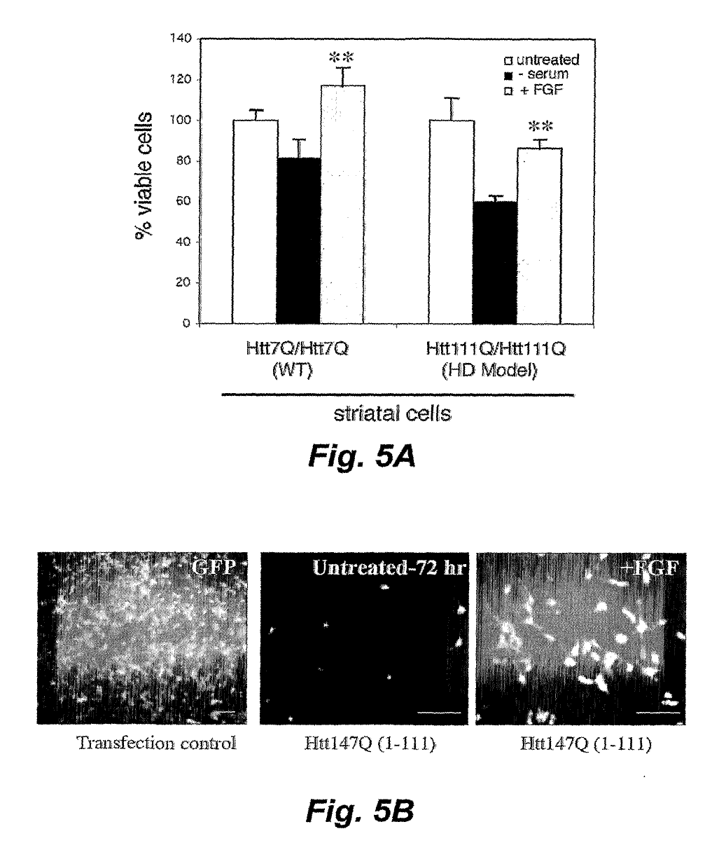

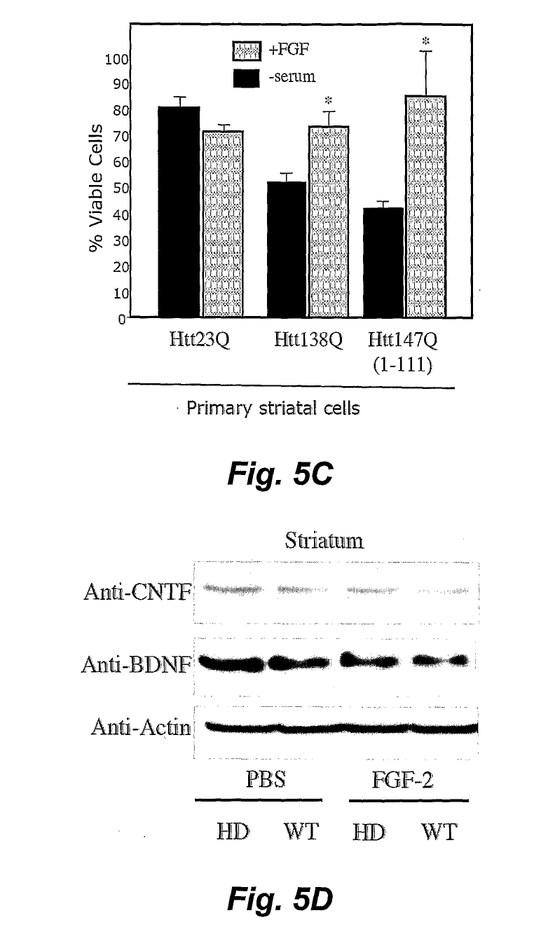

Growth factors such as BDNF, ciliary neurotrophic factor (CNTF) and insulin-like growth factor-1 (IGF-1) inhibit mutant Htt-induced cell death in primary striatal cultures (Humbert et al. (2002) Dev Cell 2: 831-837; Saudou et al. (1998) Cell 95: 55-66) or toxin-induced HD mouse models (Maksimovic et al. (2002) Vojnosanit Pregl 59: 119-123), but FGF-2 has not been evaluated in genetic models of HD. To test if FGF-2 is neuroprotective, we used immortalized striatal wild type and mutant cells derived from HD knock-in mice expressing Htt with expanded, 111-polyglutamine repeats (Trettel et al. (2002) Hum Mol Genet. 9: 2799-2809). Withdrawal of serum from STHtt^{Q111/Q111} cell cultures caused death of 44% of cells after 24 h (FIG. 5a).

This cell death had features of apoptosis, including nuclear fragmentation and activation of caspase-3-like activity (data not shown). Treatment of STHtt^{Q7/Q7} and mutant STHtt^{Q111/Q111} cells with FGF-2 (10 ng/mL) significantly increased cell survival (FIG. 5a). In contrast, FGF-2 did not increase cell proliferation, as measured by cell number and BrdU incorporation (data not shown).

Next, we evaluated whether cell death induced by expression of mutant Htt (full-length Htt or N-terminal polyQ Htt fragments) in primary striatal cultures was rescued by FGF-2 treatment. Using GFP as a positive control, we found we could transfect primary striatal neurons by electroporation with greater than 50% transfection efficiency (FIG. 5b, left panel). Both full-length Htt138Q-GFP and an N-terminal fragment (Htt147Q (1-110)) produced cell death, affecting 38% and 48% of cells, respectively, by 48 hours. Treatment with FGF-2 (10 ng/mL) resulted in a significant decrease in mutant Httinduced neuronal death (FIG. 5c). Again, FGF-2 did not increase cell proliferation as measured by cell number and BrdU incorporation (data not shown).

The mechanisms by which FGF-2 stimulates neurogenesis and affords neuroprotection are not known. Because BDNF stimulates striatal neurogenesis (Chmielnicki et al. (2004) J Neurosci 24: 2133-2142) and may be depleted in HD striatum (Zuccato et al. (2001) Science 293: 493-498), we evaluated whether FGF-2 alters BDNF expression in HD transgenic R6/2 mouse striatum. Western blot analysis of striatal lysates derived from FGF-2-treated HD transgenic R6/2 and control mice (FIG. 5d) showed no FGF-2-induced increase in the expression of either BDNF or CNTF, another trophic factor that is protective in some models of HD (Alberch et al. (2004) Prog Brain Res 146: 195-229.

FGF-2 Reduces Aggregate Formation and Corrects Signaling Defects in HD Transgenic R6/2 Mice

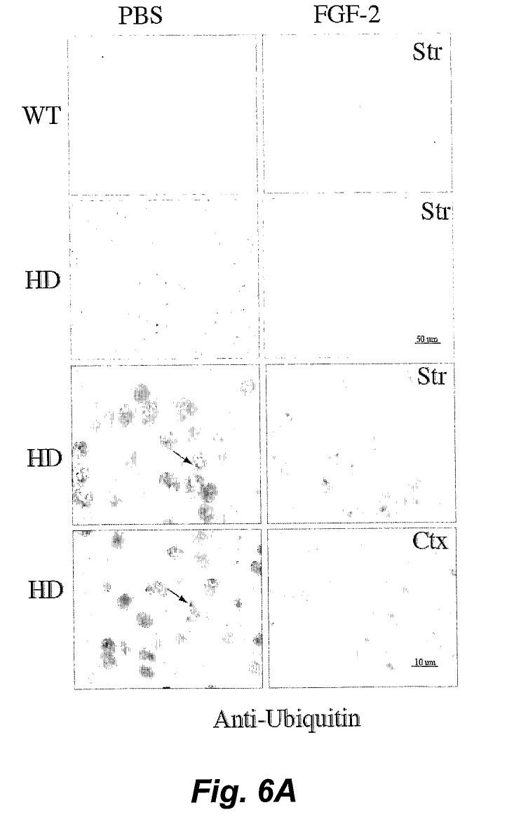

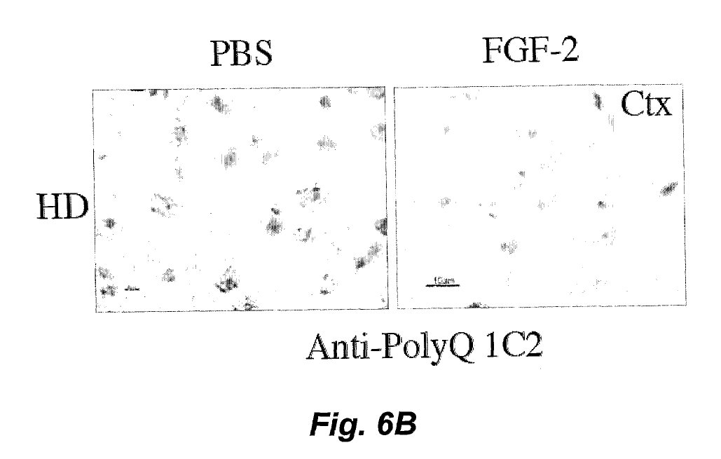

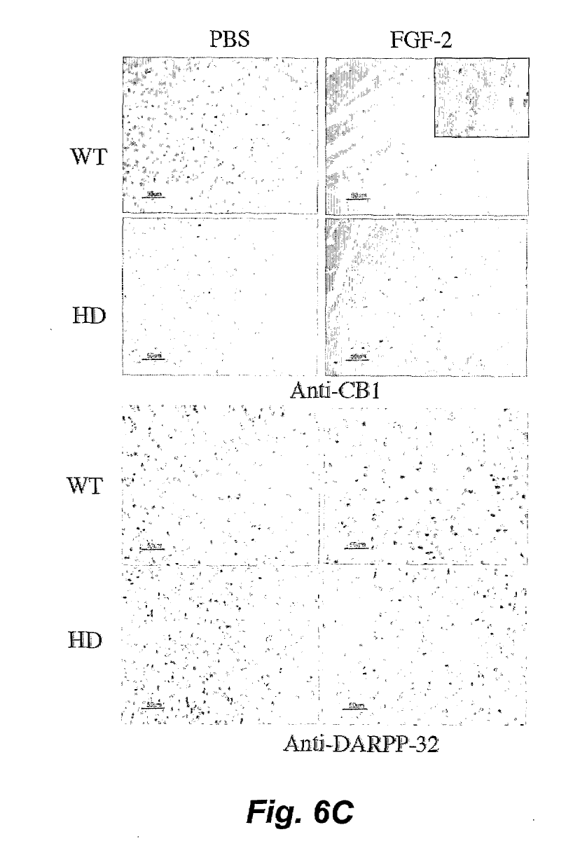

Because alterations in cellular signaling pathways and the hallmark formation of nuclear and cytoplasmic aggregates are well-characterized in HD transgenic R6/2 mice, we evaluated if FGF-2 treatment corrected any of these cellular changes. There is an early and dramatic increase in Htt-immunoreactive intraneuronal aggregates in R6/2 mice. Treatment with FGF-2 resulted in a significant reduction in Htt-positive striatal and cortical, nuclear and perinuclear aggregates—detected by antibodies against ubiquitin-positive nuclear inclusions (FIG. 6a) or against the polyglutamine tract of Htt (FIG. 6a)—at 11 weeks of age. Quantification of the ubiquitin positive nuclear inclusions (Table 1) demonstrated FGF-2 treatment reduced inclusions by 17% in the striatum (74+/−4% to 57+/−4%) and 16% in the cortex (82+/−4% to 66+/−1%).

TABLE 1
Ubiquitin positive intranuclear inclusions in
R6/2 FGF-2 treated
(n = 3) and untreated mice (n = 3).

| | | | | | |
|---|---|---|---|---|---|
| Nuclei with | | % | Nuclei with | | |
| inclusions | Total | Inclusions | inclusions | Total | % Inclusions |

Striatum

| | | | | | |
|---|---|---|---|---|---|
| 550 | 1010 | 54% | 709 | 1003 | 71% |
| 600 | 980 | 61% | 750 | 950 | 78% |
| 530 | 960 | 55% | 763 | 1043 | 73% |

Cortex

| | | | | | |
|---|---|---|---|---|---|
| 663 | 1002 | 66% | 803 | 997 | 81% |
| 501 | 1078 | 65% | 766 | 965 | 79% |
| 646 | 999 | 65% | 892 | 1036 | 86% |

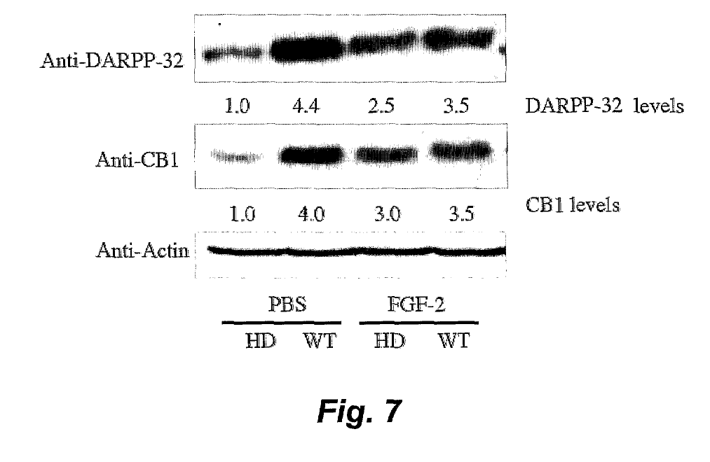

CB 1 cannabinoid receptors are down-regulated at the mRNA and protein levels in post-mortem HD brain tissue and HD transgenic R6/2 mice (Glass et al. (2004) Neuroscieizce 123: 207-212; Lastres-Becker et al. (2003) Curr Drug Target CNS Neurol Disord 2: 335-347; Lastres-Becker et al. (2002) Brain Res 929: 236-242; Lastres-Becker et al. (2001) Neuroreport 12: 2125-2129; Denovan-Wright et al. (2000) Neuroscience 98: 705-713; Glass et al. (1993) Neuroscience 56: 523-527). CB 1 receptors have been implicated in FGF-2-induced axonal growth (Williams et al. (2003) J Cell Biol 160: 481-486), and also regulate adult neurogenesis (Rueda et al. (2002) J Biol Chem 277: 46645-46650). Moreover, cannabinoids are neuroprotective in a variety of cerebral injury models (Mechoulam et al. (2002) Sci STKE 2002: RE5; Mechoulam et al. (2002) Chem Phys Lipids 121: 35-43; Mechoulam et al. (2002) Prostaglandins Leukot Essent Fatty Acids 66: 93-99). Therefore, we evaluated if FGF-2 treatment modified expression of CB 1 receptors in HD transgenic R6/2 mouse striatum. CB 1 receptor levels were reduced significantly in the striatum of HD transgenic R6/2 mice (FIG. 6c), but FGF-2 treatment restored CB 1 receptor expression (FIG. 6c; FIG. 7).

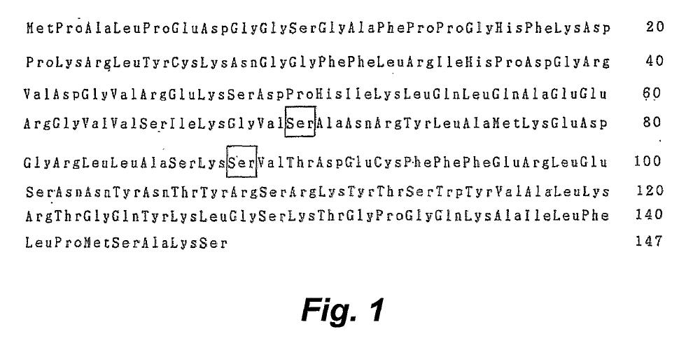

The mutation in HD transgenic R6/2 mice also downregulates the dopamine- and cAMP-regulated 32 kDa-phosphoprotein, DARPP-32 (Bibb et al. (2000) Proc. Natl. Acad. Sci.: USA: 97: 6809-6814; van Dellen et al. (2000) Neuroreport 11: 3751-3757). DARPP-32 is normally enriched in prefrontal cortex and striatum (Id.), where it participates in dopamine and serotonin signaling. We found that DARPP-32 levels were reduced by 50% in the striatum of HD transgenic R6/2 mice (FIG. 6c). Western blotting (Supplementary FIG. 1) and immunostaining also showed that FGF-2 increased in DARPP-32 expression in 11 week-old HD transgenic R6/2 mice (FIG. 6c).

### Discussion

The major findings we report are that FGF-2 stimulates neurogenesis, provides neuroprotection, and extends lifespan in a transgenic mouse model of HD. Neurogenesis, detected by BrdU labeling and DCX expression, was increased under basal conditions, and stimulated to a greater extent by FGF-2, in SVZ but not DG of HD transgenic R6/2 mice. The increase in neurogenesis was associated with migration of nascent neurons into the affected striatum, where they assumed phenotypic features of medium spiny neurons, the principal striatal cell type lost in HD, and extended processes to the globus pallidus, where medium spiny neurons normally project. Neuroprotection was observed both and, and was manifested by reductions in neuronal death and protein aggregate formation, and restoration towards normal of CB 1 cannabinoid receptor and DARPP-32 protein expression, improved neurological function and prolonged survival. Whether neurogenesis, direct neuroprotection or both contribute to the improvement in neurological function and longevity that we observed cannot be resolved based on present data. FGF-2 may also have peripheral (non-brain) effects that contribute to longevity. However, the effect of FGF-2 in HD transgenic R6/2 mice raises the possibility that FGF-2 or a drug that recapitulates one or more of its effects may provide a prototype for the treatment of HD.

Despite recent advances in understanding the molecular pathogenesis of HD, clinical measures to slow or arrest disease progression are lacking. Growth factors have received considerable attention in preclinical studies, however, and several—including nerve growth factor, BDNF, neurotrophins 3 and 4, glial cell-derived neurotrophic factor, neurturin and CNTF—have shown some benefit in excitotoxic models of HD when administered directly or by gene or cell therapy (reviewed in Alberch et al. (2004) Prog Brain Res 146: 195-229). Genetic models of HD have been studied less extensively in this regard, but BDNF expression is decreased in transgenic murine (Zuccato et al. (2001) Science 293: 493-498) and human (Ferrer et al. (2000) Brain Res 866: 257-261) HD, and both BDNF and CNTF rescue cultured striatal neurons from death induced by transfection with mutant Htt³⁰. FGF-2 treatment in our studies did not alter BDNF levels in the striatum and therefore promotes neurogenesis and survival independent of BDNF levels. Prolonged survival has been reported in HD transgenic mice treated with a dominant-negative inhibitor of caspase-1 (Ona et al. (1999) Nature 399: 263-267), creatine (Ferrante et al. (2000) J Neurosci 20: 4389-4397), minocycline (Chen et al. (2000) Nat Med 6: 797-801), dichloroacetate (Andreassen et al. (2001) Ann Neurol 50: 112-117), -lipoic acid (Andreassen et al. (2001) Neuroreport 12: 3371-3373), cystamine (Dedeoglu et al. (2002) J Neurosci 22: 8942-8950; Karpuj et al. (2002) Nat Med 8: 143-149), coezyme Q10 (Ferrante et al. (2002) J Neurosci 22: 1592-1599), remacemide (Id.), the antioxidant BN8245 1(Klivenyi et al. (2003) J Neurochem 86: 267-272), histone deacetylase inhibitors (Hockly et al. (2003) Proc Natl Acad Sci USA 100: 2041-2046; Ferrante et al. (2003) J Neurosci 23: 9418-91427), rapamycin (Ravikumar et al. (2004) Nat Genet. 36: 585-595) or the disaccharide trehalose (Tanaka et al. (2004) Nat Med 10:148-154). In these studies, the increase in mean survival has ranged from 10 to 20%. The mechanisms through which some of these treatments may operate have been inferred from their actions in other systems, but in other cases are unclear. Additional measures that prolong survival in HD transgenic mice include environmental enrichment (Spires et al. (2004) J Neurosci 24: 2270-2276; Carter et al. (2005) Mov Disord 15: 925-937; Hockly et al. (2002) Ann Neurol 51: 235-242), the anti-excitotoxin riluzole (Schiefer et al. (2002) Mov Disord 17: 748-757) and the antidepressant paroxetine (Duan et al (2004) Ann Neurol 55: 590-594), which also have in common the ability to stimulate neurogenesis (Kempermann et al. (1997) Nature 386: 493-495; Katoh-Semba et al. (2002) FASEB J 16: 1328-1330; Santarelli et al. (2003) Science 301: 805-809), although this has not been shown to be the basis for their protective effect in HD.

FGF-2 is neuroprotective in a variety of neurological disease models (reviewed in Reuss et al. (2003) Cell Tissue Res 313: 139-157), including global (Nakata et al. (1993) Brain Res 605: 354-356) and focal (Bethel et al. (1997) Stroke 28: 609-615; discussion 615-616) cerebral ischemia, kainate-induced seizures (Liu et al. (1993) Brain Res 626: 335-338) and MPTP-mediated parkinsonism (Otto et al. (1990) J Neurosci 10: 1912-1921). FGF-2 is expressed in substantia nigra, striatum and globus pallidus of human brain, and FGF receptor expression is increased in HD (Tooyama et al. (1993) Brain Res 610:1-7). In the quinolinic acid model of HD in rats, FGF-2 attenuates changes in cytochrome oxidase (Maksimovic et al. (2001) Vojnosanit Pregl 58: 237-242) and nitric oxide synthase (Maksimovic et al. (2002) Vojizosanit Pregl 59: 119-123) activity, but there is little other prior evidence to connect FGF-2 with HD. The mechanisms through which FGF-2 produced neuroprotection in our HD transgenic R6/2 mice may relate to the Akt signaling pathway. FGF-2 activates a range of signal transduction pathways (reviewed in Ensoli et al. (2003) The fibroblast growth factors. in The Cytokine Handbook, Vol. 2 (eds. Thomson, A. W. & Lotze, M. T.) 747-781 (Elsevier, London)), among which the phosphatidylinositol 3′-kinase (PI3K)/Akt pathway may be especially prominent in promoting cell survival. Notably, Akt signaling has also been implicated in the protective effect of IGF-1 in cultured cells expressing mutant Htt (Humbert et al. (2002) Dev Cell 2: 831-837).

The ability of FGF-2 to stimulate neurogenesis in the adult brain is well established. FGF-2 increases the proliferation of neuronal precursors (Gensburger et al. (1987) FEBS Lett 217: 1-5) and intraventricular infusion of FGF-2 enhances the proliferation and migration of neuronal precursors in the SVZ (Kuhn et al. (199) J Neurosci 17: 5820-5829). Moreover, injury-induced neurogenesis is impaired in FGF-2-knockout mice, and is restored by replacement of FGF-2 using a herpesvirus amplicon vector (Yoshimura et al. (2001) Proc Natl Acad Sci USA 98: 5874-5879). As mentioned above in connection with FGF-2-mediated neuroprotection, the molecular basis for FGF-2-induced neurogenesis remains speculative, although cytoproliferative actions of FGF-2 in other systems have been ascribed to activation of MEK/ERK pathways (Ensoli et al. (2003) The fibroblast growth factors. in The Cytokine Handbook, Vol. 2 (eds. Thomson, A. W. & Lotze, M. T.) 747-781 (Elsevier, London)), and MEK/ERK signaling has also been implicated in neurogenesis induced by NT-3 and BDNF (Bamabe-Heider and Miller (2003) J Neurosci 23: 5149-5160).

The increased neurogenesis that we observed in HD transgenic R6/2 mice represents another illustration of the emerging theme that neurogenesis is stimulated in neurological diseases, possibly as an adaptive response directed towards neuronal replacement. Examples include HD (Curtis et al (2003) Proc. Natl. Acad. Sci., USA, 100: 9023-9027) and Alzheimer's disease (AD) ( )Jin et al. (2004) Proc. Natl. Acad. Sci., USA, 101: 343-347 in humans, as well as animal models of Parkinson's disease (Zhao et al. (2003) Proc. Natl. Acad. Sci., USA, 100, 7925-7930), global (Liu et al. (1998) J Neurosci 18: 7768-7778) and focal (Jin et al. (2001) Proc. Natl. Acad. Sci., USA, 98: 4710-4715) cerebral ischemia and epilepsy (Parent et al. (1997) J. Neurosci. 17: 3727-3738). Injury-induced neurogenesis shows both similarities and differences across disease models, but the examples cited above demonstrate that it can be precipitated by either acute or chronic and by focal or diffuse brain pathology. There appears to be some degree of regional specificity in the propensity of brain lesions to evoke neurproliferative responses in DG or SVZ. Thus, we observed increased BrdU labeling in the juxtastriatal SVZ but not in the DG of our HD transgenic R6/2 mice, whereas neuronal precursors in DG appear to be mobilized preferentially in disorders that prominently affect the hippocampus, such as global cerebral ischemia (Liu et al. (1998) J Neurosci 18, 7768-7778) and AD (Jin et al. (2004) Proc. Natl. Acad. Sci., USA, 101: 343-347). Where injury occurs at a distance from the brain's principal neuroproliferative zones, as best exemplified in focal ischemia, but also true for animal models of HD (this report) and AD, newborn neurons migrate from their sites of origin into affected brain areas. In ischemia affecting the striatum (Parent et al. (2002) Ann. Neurol. 52: 802-813) and in HD transgenic R6/2 mice, new neurons migrating into the striatum differentiate towards a phenotype resembling that of the dead or injured cells. It is noted that in the HD transgenic R6/2 mice, the new neurons extend projections to anatomically appropriate targets. The contribution of neurogenesis to functional recovery from brain injury is difficult to ascertain, because as in the present study, treatments that stimulate neurogenesis may have additional, potentially beneficial effects on cell function. However, one recent report showed that blocking neurogenesis by cerebral x-irradiation in gerbils impaired recovery from global cerebral ischemia (Raber et al. (2004) Ann Neurol 55, 381-389), suggesting that neurogenesis contributes to recovery.

It is believed that the beneficial effect of FGF2 might be optimized by, for example, earlier onset of administration, alterations in dosage or route of delivery, or combining FGF2 with one or more of the numerous growth factors or drugs (discussed above) that yield benefit in animal models of HD, enhance neurogenesis, or both.

### Methods:

Tissue Culture, Western Blot, and Immunohistochemistry:

Supplementary methods contain these experimental procedures.

R6/2 Transgenic and Wild-Type Mice.

All animal experiments were performed according to procedures approved by the Institutional Animal Care and Use Committee. Heterozygous Htt exon-1-trangenic mice of strain R6/2 (145 CAG repeats) were obtained from the Jackson Laboratory (Bar Harbor, Me.). Animals were genotyped by PCR of tail-tip DNA, and CAG repeat size was determined. Wild-type (n=20) and R6/2 (n=20) were separated into equal FGF-2 (Chemicon; GF003) or vehicle (PBS) treatment groups according to Hockly et al. (2003) Brain Res Bull 61: 469-479. The treatment groups contained the same number of females and males. FGF-2 (250 ng/animal) was administered subcutaneously twice daily, three days per week, until animals were used for immunohistochemistry or survival studies staring at 59 days of age. The BrdU+ counts and survival studies were carried out in a double blind manner.

Behavioral Analysis.

Disease progression and survival status were monitored daily; the first day on which limb tremors were detected was designated the day of disease onset. Rotarod performance (accelerating regime) and lifespan were analyzed as previously described (Hockly et al. (2003) Proc Natl Acad Sci USA 100: 2041-2046; Hockly et al. (2003) Brain Res Bull 61: 469-479; Ferrante et al. (2003) J Neurosci 23: 9418-91427).

Statistical Analysis.

Statistical comparisons of rotarod, weight data, and histology data are compared by ANOVA. Survival data were analyzed using Kaplan-Meier survival curves (n=10 per treatment group).

BrdU Administration.

FGF-2 and vehicle was administered for three weeks prior to BrdU treatment starting at 59 days. BrdU (Sigma, St. Louis, Mo., USA) was dissolved in saline and given as two intraperitoneal doses of 50 mg/kg each, spaced 8 h apart per day, for three days and then mice were killed 24 h or 7 days later.

Supplementary Methods:

In Vitro and in Vivo Cell Culture Experiments.

Conditionally immortalized wild type STHtt^{Q7/Q7} striatal neuronal progenitor cells expressing endogenous normal Htt and homozygous mutant STHtt^{Q111/Q111} striatal neuronal progenitor cell lines expressing endogenous mutant Htt with 111-glutamines, generated from Htt^{Q111/Q111} and wild type Htt^{Q7/Q7} littermate embryos. The striatal cell lines were grown at 33° C. in Dulbecco's modified Eagle's medium supplemented with 10% fetal bovine serum, 1% nonessential amino acids, 2 mM L-glutamine, and 400 g/ml G41 8 (Geneticin, Invitrogen). Primary cultures of mouse striatum were prepared as described previously (Hermel et al. (2004) Cell Death Differ 11: 424-438). The cells were resuspended at a density of 4×10⁶ cells/1001 in Amaxa nucleofector solution (Amaxa Inc., Gaithersburg, Md.) and electroporated according to the manufacturer's specifications with Hft15Q-GFP, Htt138Q-GFP, myc-Htt23Q (1-110) and myc-Htt143Q (1-110). Immunofluorescence was measured as previously described (Hermel et al. (2004) Cell Death Differ 11, 424-438). Cells were diluted with DMEM containing 10% FBS and seeded onto polylysine-coated glass chamber slides (Becton Dickinson Labware, Franklin Lakes, N.J.) at 2.5−3.5×10⁵ cells/cm². After 30 min, the medium was replaced with neurobasal A medium containing 1 mM glutaMax-1, 24.5 mM KCl and 2% B-27 (Invitrogen, CA). The cultures were incubated at 37° C. in 95% air/5% carbon dioxide at 95% humidity. Efficiency of transfection was between 75-85% by day 4 as estimated by GFP fluorescence. Cell death was measured with the WST-1 (Roche Molecular Biochemicals) or LIVE/DEAD assay (Molecular Probes) according to manufacturer's instructions. Cell numbers were counted with a hemocytometer.

Cells were lysed with RIPA lysis buffer (50 mM Tris-HCl, 150 mM NaCl, 0.1% SDS, 1% SDOC, 1% NP40; pH 8.0) and protease inhibitor Minicomplete (Roche Applied Science), sonicated (5×10 sec pulses), and clarified by centrifugation at 16,000× for 20 min at 4° C. Protein concentration was determined using the BCA method (Biorad). Lysate (50 mg) was resolved by SDS-PAGE using 4-12% Bis-Tris precast gels (Invitrogen) and transferred to PVDF membranes (Biorad) for 1 h at 25 mA or for 14 h at 120 mA. Membranes were blocked in 5% milk in TBST. Western blotting was performed with monoclonal anti-CNTF (1:100, Chemicon, MAB338), monoclonal anti-BDNF (1:250, Sigma, B5050), rabbit polyclonal anti-DARPP-32 (1:500, Chemicon, AB 1656), rabbit polyclonal anti-CB 1 (1:250, Affinity BioReagents, PA1-745), or polyclonal anti-actin (1:5000, Sigma A5441) for 2 h at room temperature or for 18 h at 4° C. Secondary anti-rabbit or anti-mouse (1:3000; Amersham Biosciences) antibody was applied for 45 min at room temperature and ECL (Amersham Biosciences) was used for detection.

Immunohistochemistry for Frozen Sections.

Sections were fixed with 4% paraformaldehyde in PBS for 1 h at room temperature, washed twice with PBS, and incubated in 2 M HCl at 37° C. for 1 h. Sections (50 μm) were cut with a cryostat and stored at −80° C. Sections were pretreated with 50% formamide, 280 mm NaCl, and 30 mM sodium citrate at 65° C. for 2 h, incubated in 2 M HCl at 37° C. for 30 min, and rinsed in 0.1 M boric acid (pH 8.5) at room temperature for 10 min. Sections were incubated in 1% H₂O₂ in PBS for 15 min, in blocking solution (2% goat serum, 0.3% Triton X-100, and 0.1% bovine serum albumin in PBS) for 2 h at room temperature, and with 2 μg/ml of mouse monoclonal anti-BrdU antibody (Roche) at 4° C. overnight. Sections were washed with PBS, incubated with biotinylated goat anti-mouse secondary antibody (1:200, Vector) for 2 h at 25° C., washed, and placed in avidin-peroxidase conjugate (Vector) solution for 1 h. The horseradish peroxidase reaction was detected with 0.05% diaminobenzidine (DAB) and 0.03% H₂O₂. Processing was stopped with H₂O, and sections were dehydrated through graded alcohols, cleared in xylene, and coverslipped in permanent mounting medium (Vector). Sections were examined with a Nikon E300 epifluorescence microscope.

Immunocytochemistry for Paraffin-Embedded Tissue.

Brains were paraffin-embedded after perfusion with saline and 4% paraformaldehyde in PBS, sectioned horizontally (8 m) on the automated rotary microtome (Leica), and deparaffinized in xylene. Antigen retrieval was carried out by microwaving sections in 10 mM citrate buffer, pH 6.0, for 2 min at 40% power in a 1100 W microwave oven. After washing, sections were incubated in 0.3% H₂O₂ in PBS for 15 min. After washing again, the sections were incubated in blocking solution (1% sheep serum, 0.1% bovine serum albumin, 0.3% Triton X-100, PBS) for 30 min. Sections were incubated in primary antibodies at 4° C. overnight, and with secondary antibodies in blocking solution at room temperature for 2 h. The primary antibodies used were affinity-purified goat polyclonal anti-DCX (1:100, Santa Cruz Biotechnology, sc8067), monoclonal anti-polyglutamine 1C2 (1:500, Chemicon), rabbit polyclonal anti-DARPP-32 (1:500, Chemicon, AB 1656), ubiquitin (1:1000, DAK0, Z0458), monoclonal anti-NeuN (1:500, Chemicon, MAB377) and rabbit polyclonal anti-CB 1 cannabinoid receptor (1:50, Oncogene, PC24 1). For DAB staining, sections were washed with PBS, incubated with biotinylated goat anti-mouse secondary antibody (1:200, Vector) for 2 h at 25° C., washed, and placed in avidin-peroxidase conjugate (Vector) solution for 1 h. The horseradish peroxidase reaction was detected with 0.05% DAB and 0.03% H₂O₂. Processing was stopped with H₂O, and sections were dehydrated through graded alcohols, cleared in xylene, and coverslipped in permanent mounting medium (Vector). Sections were examined with a Nikon E300 epifluorescence microscope. For immunofluorescence, the secondary antibodies were Cy₃-conjugated donkey anti-mouse IgG or anti-rabbit IgG (1:250, Jackson ImmunoResearch) and FITC-conjugated donkey anti-goat IgG (1:100, Jackson ImmunoResearch). Sections were mounted with Vectashield (Vector), and fluorescence signals were detected with a Nikon E800 microscope at excitation/emission wavelengths of 535/565 nm (rhodamine, red) and 470/505 (FITC, green). Results were recorded with a Magnifire digital camera (ChipCoolers). For confocal microscopy, a Nikon PCM-2000 laser-scanning confocal microscope and Simple PCI imaging software (Compix) were used.

### Example 2

### Increased Striatal and FGF-Induced Nigral Neurogenesis in the Acute MPTP Model of Parkinson's Disease

In response to injury, endogenous precursors in the adult brain can proliferate and generate new neurons, which may have the capacity to replace dysfunctional or dead cells. Although injury-induced neurogenesis has been demonstrated in animal models of stroke, Alzheimer's disease and Huntington's disease (HD), studies of Parkinson's disease (PD) have produced conflicting results. In this study, we investigated the ability of adult mice to generate new neurons in response to the parkinsonian toxin 1-methyl-4-phenyl-1,2,3,6-tetrahydropyridine (MPTP), which causes selective degeneration of nigrostriatal dopamine neurons. MPTP lesions increased the incorporation of 5-bromo-2′-deoxyuridine-5′-monophosphate (BrdU), as well as the number of cells that co-expressed BrdU and the immature neuronal marker doublecortin (Dcx), in two neuroproliferative regions—the subgranular zone of the dentate gyrus (DG) and the rostral subventricular zone (SVZ). BrdU-labeled, Dcx-expressing cells were not found in the substantia nigra (SN) of MPTP-treated mice, where neuronal cell bodies are destroyed, but were present in increased numbers in the striatum, where SN neurons lost in PD normally project. Fibroblast growth factor-2 (FGF2), which enhances neurogenesis in a mouse model of HD, also increased the number of BrdU/Dcx-immunopositive cells in the SN of MPTP-treated mice. Thus, MPTP-induced brain injury increases striatal neurogenesis and, in combination with FGF2 treatment, also stimulates neurogenesis in SN.

### Introduction

Conflicting results have been obtained regarding whether animal models of PD stimulate endogenous neurogenesis, as reported for animal models of certain other neurodegenerative disorders. Rat substantia nigra (SN) has been reported to contain neuronal progenitor cells, identified by labeling with bromodeoxyuridine (BrdU) and expression of immature or mature neuronal lineage markers (Lie et al. (2002) J Neurosci 22:6639-6649). Mouse SN has also been reported to contain a small number of BrdU-positive, dopaminergic cells thought to originate in the SVZ (Zhao et al. (2003) Proc. Natl. Acad. Sci., USA, 100: 7925-7930). However, other laboratories have reported that they were unable to replicate these findings, calling into question the ability of endogenous neurogenesis in the adult to generate SN dopaminergic neurons, at least in the absence of disease-related stimulation (Frielingsdorf et al. (2004) Proc. Natl. Acad. Sci., USA, 101:10177-10182).

The discordant results of studies to date suggest that the relationship between neurogenesis and parkinsonism may be complex and that differences in the animal models employed or in the severity or duration of disease may explain some of the disparities. In this study, we investigated the effect of acute N-methyl-4-phenyl-1,2,3,6-tetrahydropyridine hydrochloride (MPTP) administration on neurogenesis in the adult mouse brain, using BrdU to label proliferating cells and cell type-specific antibodies to characterize their phenotype. The results indicate that acute MPTP treatment promotes neurogenesis in the SGZ, SVZ and striatum, and, after administration of FGF2, in the SN. Therefore, MPTP-induced parkinsonism appears to stimulate neurogenesis in a manner that could contribute to functional replacement of nigrostriatal circuitry.

### Materials and Methods

MPTP Administration

MPTP-HCl (20 mg/kg free base, Sigma, St. Louis, Mo.) dissolved in saline was administered intraperitoneally to male C57BL/6 mice (8-week-old, Charles River Laboratories) every 2 h for four doses. Mice used as controls received an equivalent volume of saline. Experimental protocols were in accordance with the National Institutes of Health Guidelines for Use of Live Animals and were approved by the Animal Care and Use Committee at the Buck Institute.

FGF2 and BrdU Administration

Each mouse was intraperitoneally injected with either recombinant human fibroblast growth factor 2 (38 μg/kg, Chemicon, Temecula, Calif.) in PBS or PBS alone for 8 d (days 0-7 after MPTP administration). BrdU (50 mg/kg; Sigma, St. Louis) dissolved in saline was given intraperitoneally twice daily, at 8-h intervals, on consecutive days (days 1-3, 7-9, or 1-14 after MPTP administration). Mice were killed on day 14 or 21 after MPTP administration.

BrdU-Immunopositive Cell Counting

Brains were removed after perfusion with saline and 4% paraformaldehyde in phosphate buffered saline (PBS). Adjacent 50 g/m sections were cut with a cryostat and stored at −80° C. Sections were pretreated with 50% formamide, 280 mM NaCl, and 30 mM sodium citrate at 65° C. for 2 hr, incubated in 2 M HCl at 37° C. for 30 min, and rinsed in 0.1 M boric acid, pH 8.5, at room temperature for 10 min. Sections were incubated in 1% H₂O₂ in PBS for 15 min, in blocking solution (2% goat serum, 0.3% Triton X-100, and 0.1% bovine serum albumin in PBS) for 2 hr at room temperature, and with 2 μg/ml of mouse monoclonal anti-BrdU antibody (Roche) at 4° C. overnight. Sections were washed with PBS, incubated with biotinylated goat anti-mouse secondary antibody (Vector; 1:200) for 2 hr at 25° C., washed, and placed in avidin-peroxidase conjugate (Vector) solution for 1 hr. The horseradish peroxidase reaction was detected with 0.05% diaminobenzidine (DAB) and 0.03% H₂O₂. Processing was stopped with H₂O, and sections were dehydrated through graded alcohols, cleared in xylene, and coverslipped in permanent mounting medium (Vector).

BrdU-positive cells in SGZ and SVZ were counted blindly in five to seven DAB-stained, 50 μm coronal sections per mouse, spaced 200 μm apart. Cells were counted under high-power (200×) on a Nikon E300 microscope with a Magnifire digital camera and the image was displayed on a computer monitor. Results were expressed as the average number of BrdU-positive cells per section.

Fluorescence Immunohistochemistry

Sections were fixed with 4% paraformaldehyde in PBS for 1 hr at room temperature, washed twice with PBS, and incubated in 2 M HCl at 37° C. for 1 hr. After washing again, sections were incubated with blocking solution, then with primary antibodies at 4° C. overnight, and with secondary antibodies in blocking solution at room temperature for 2 hr. The primary antibodies used were mouse monoclonal anti-BrdU (Roche, Indianapolis, Ind.; 2 μg/ml), sheep polyclonal anti-BrdU (Biodesign, Saco, Me.; 25 μg/ml), mouse monoclonal anti-Ki67 antigen (Novocastra Laboratories Ltd; 1:50), mouse monoclonal anti-neuronal nuclear antigen (NeuN) (Chemicon, Temecula, Calif.; 1:200), affinity-purified goat polyclonal anti-doublecortin (Dcx) (Santa Cruz Biotechnology; 1:200), mouse monoclonal anti-III-tubulin (Caltag Laboratories, Burlingame, Calif.; 1:400), mouse monoclonal anti-glial fibrillary acidic protein (GFAP) IgG (Sigma; 1:400), rat anti-mouse CD11b (Serotec Inc. Raleigh, N.C.; 1:50), mouse monoclonal anti-2′,3′-cyclic nucleotide 3′-phosphodiesterase (CNPS) (Chemicon, Temecula, Calif.; 1:500), rat anti-polysialic acid-neural cell adhesion molecule (PSA-NCAM) (BD Biosciences; 1:100), and rabbit polyclonal anti-tyrosine hydroxylase (TH) (Chemicon, Temecula, Calif.; 1:200). The secondary antibodies were rhodamine-conjugated rat-absorbed donkey anti-mouse IgG, rhodamine-conjugated rat-absorbed donkey anti-sheep IgG (Jackson ImmunoResearch, West Grove, Pa.; 1:200), fluorescein isothiocyanate (FITC)-conjugated goat anti-mouse IgG, FITC-conjugated pig anti-goat IgG, FITC-conjugated goat anti-rat IgG (Vector, Burlingame, Calif.; 1:200), and FITC-conjugated goat anti-rabbit IgG (Vector, Burlingame, Calif.; 1:200). Sections were mounted with Vectashield (Vector), and fluorescence signals were detected with a Nikon PCM-2000 laser-scanning confocal microscope and Simple PCI imaging software (Compix) were used.

Statistical Analysis

All data are expressed as mean±S.E. for the number (n) of independent experiments performed. Differences among the means for all experiments described were analyzed using one- or two-way analysis of variance. Newman-Keuls post-hoc analysis was employed when differences were observed by analysis of variance testing (p<0.05).

### Results

### Neurogenesis in the DG and Rostral SVZ is Increased Following Acute MPTP Administration

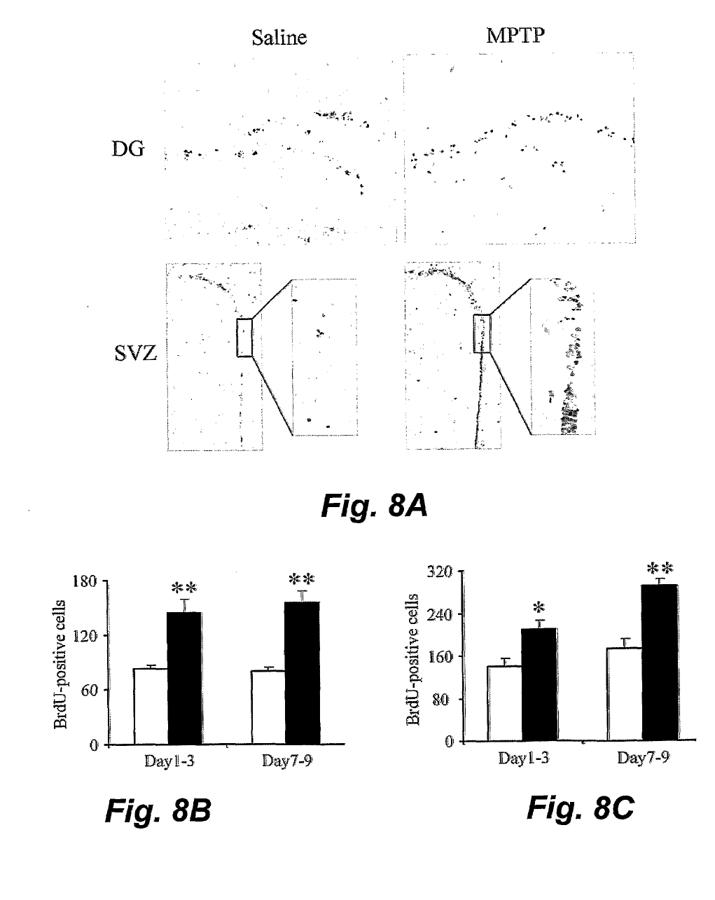

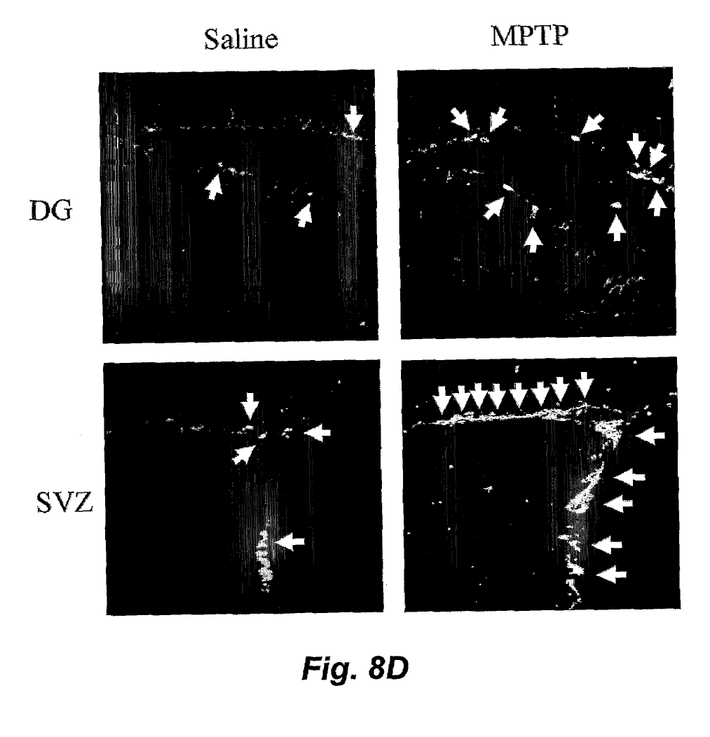

Because neurogenesis persists in the adult mammalian brain and can be regulated by physiological and pathological events, we investigated its possible involvement in the brain's response to MPTP-induced neurotoxicity. Mice received MPTP (four intraperitoneal injections per day of 20 mg/kg body weight at 2-hr intervals) and proliferating cells were labeled with BrdU (2 injections per day of 50 mg/kg body weight at 8-hr intervals) over 3-day periods, beginning on day 1 or 7 following the last MPTP injection. Mice were sacrificed 2 weeks after the last MPTP injection. Acute MPTP administration increased the incorporation of BrdU into cells in two neuroproliferative regions—the subgranular zone of the DG and the rostral SVZ (FIG. 8A). To quantify changes in BrdU labeling following MPTP injection, we counted the number of BrdU-reactive nuclei in brain sections from saline- vs. MPTP-injected mice. As shown in FIGS. 1B and 1C, acute MPTP administration resulted in approximately 70-90% and 36% increases in the numbers of BrdU-labeled cells in the DG and SVZ, respectively, as compared with saline-injected controls at days 7-9.

Relationship between BrdU Labeling and Dcx Immunoreactivity

To investigate whether BrdU labeling following acute MPTP challenge correlates with labeling of neuronal precursors in the proliferation zones, we double-labeled brain sections with antibodies against BrdU and the developmentally-regulated marker doublecortin (Dcx), a microtubule-associated protein found in the soma and processes of migrating neurons during development (Gleeson et al. (1999) Neuron 23:257-271) and in lesion-induced adult neurogenesis (Magavi et al. (2000) Nature 405:951-955). Compared to saline controls, there were more BrdU-labeled cells expressing Dcx in both the DG and the SVZ of the MPTP-injured brains (FIG. 8D). BrdU-labeled cells co-expressed both Dcx and proliferating cell nuclear antigen suggesting that they were nascent neurons.

MPTP Induces SVZ and Striatal Neurogenesis

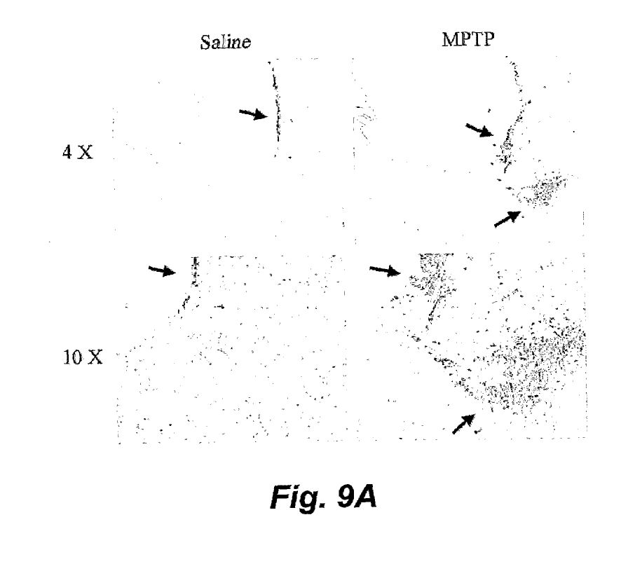

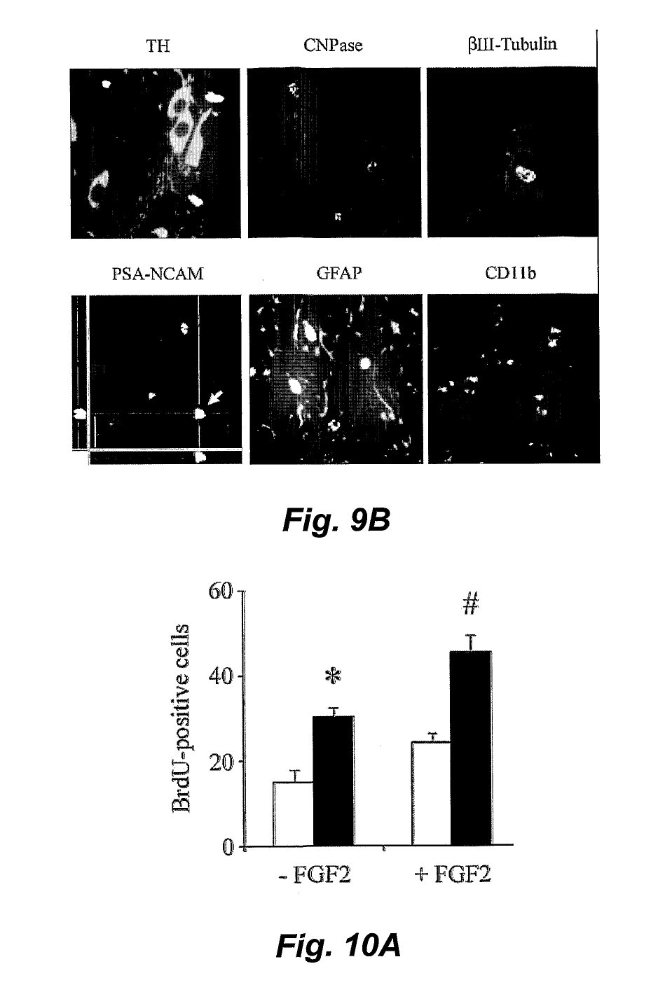

Although pathological processes can enhance neurogenesis in the adult brain, the fate of the newborn neurons that are produced and their role in brain repair are not well understood. To determine whether acute MPTP-induced neuronal proliferation is associated with migration of nascent neurons from proliferation zones toward the injury site, we mapped the migration of cells labeled by cell proliferation markers and antibodies against neuronal marker proteins for up to 3 weeks following the last MPTP administration. In the normal adult brain, Dcx is expressed in the SVZ and rostral migratory stream (RMS), but only in rare, single cells in the striatum. We found a similar pattern of expression in our saline-treated mice, whereas Dcx-labeled cells were abundant in the striatum of MPTP-treated mice (FIG. 9A).

Acute MPTP does not Increase Neurogenesis in Substantia Nigra

Because neuronal stem or progenitor cells from the adult SN can give rise to neurons after transplantation (Lie et al. (2002) J Neurosci 22:6639-6649), we hypothesized that MPTP-induced death of dopaminergic neurons in the SN might stimulate endogenous neurogenesis in this region. Proliferating cells were labeled by daily injections of BrdU for 14 consecutive days, at which time there was an increase in the number of BrdU-labeled cells in the SN of MPTP-treated compared with saline-treated mice, from 15.0±2.8 to 30.3±2.3 (n=4; p<0.01). Sections were screened for newly generated neurons or astrocytes by staining for (a) BrdU or the cell-cycle marker Ki67, (b) neuronal or glial markers (NeuN, βIII-tubulin, CNPS, CD11b, GFAP, PSA-NCAM) and (c) a dopaminergic marker (TH). Although BrdU co-localized with the immature neuronal marker polysialylated (embryonic) neural cell-adhesion molecule (PSA-NCAM) in some cells (FIG. 9B), none of the newly generated cells in SN expressed NeuN, III-tubulin, CNPS, CD11b, GFAP or TH.

FGF2 Stimulates Neurogenesis in MPTP-Treated Substantia Nigra

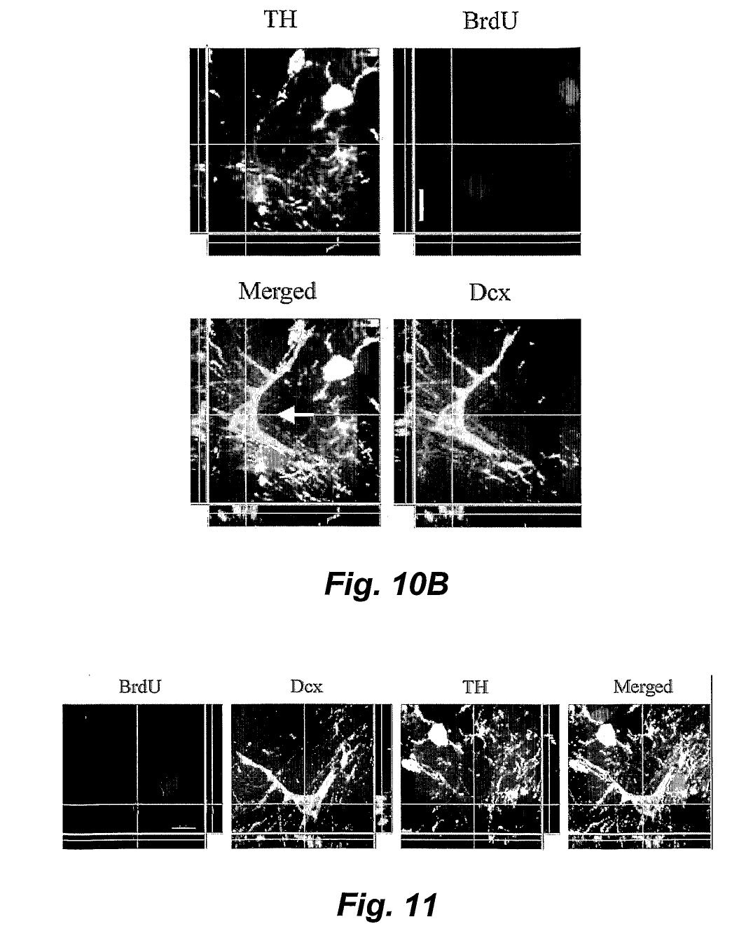

Fibroblast growth factor 2 (FGF2) has been shown to stimulate both the differentiation and survival of post-mitotic cells as well as being a proliferative factor for non-differentiated cells in the nervous system. To test whether FGF2 can stimulate neurogenesis following MPTP treatment in vivo, FGF2 was injected intraperitoneally for 10 d and BrdU for 14 d following acute MPTP administration, and mice were killed 1 week later. As shown in FIG. 3A, the number of BrdU-immunopositive cells in SN increased after FGF2 administration. Brain sections from the SVZ and SN of FGF2- and BrdU-treated mice, taken 1 week after treatment, were immunostained for BrdU and for markers of mature and immature neurons. These studies showed BrdU-immunopositive cells that coexpressed Dcx in the SN, suggesting that FGF2 can increase the number of newborn neurons in the SN following MPTP-induced injury (FIG. 10B).

### Discussion

We report that acute administration of the neurotoxin MPTP, which produces a syndrome that resembles PD in humans, stimulates neurogenesis in the adult mouse brain. Neurogenesis was identified by the occurrence within a single cell of BrdU labeling, suggesting recent provenance, and Dcx expression, establishing neuronal lineage. BrdU may also label injured cells undergoing DNA repair, but the MPTP-induced increase in labeling that we observed was in the brain's classic neuroproliferative regions (DG and SVZ), rather than in the SN, where nuclei of MPTP-damaged cells reside. In addition to its effect on DG and SVZ neurogenesis, MPTP increased the number of new (Dcx-immunoreactive) neurons in the striatum and, following FGF2 treatment, in the SN. Whether these striatal and nigral neurons arose from local progenitors or migrated from DG or SVZ cannot be resolved by the present data. However, the SVZ is immediately adjacent to the striatum, and appears to provide the new neurons that migrate there in animal models of other cerebral disorders, including stroke (Arvidsson et al. (2002) Nat Med 8:963-970, 2002; Jin et al. (2003) Mol Cell Neurosci 24:171-189) and HD (Ellerby et al. (2005) Proc. Natl. Acad. Sci., USA, 102: 18189-18194).

Previous work on neurogenesis in PD has produced conflicting results. As to whether endogenous neurogenesis occurs in the normal adult SN, the adult rat SN has been reported by one group to contain progenitor cells, identified by labeling with BrdU, that give rise exclusively to glia in situ; however, when cultured in the presence of FGF2 or FGF8 in vitro or transplanted into the dentate hilus in vivo, these progenitors produced cells that expressed immature (βIII-tubulin) or mature (NeuN) neuronal markers (Lie et al. (2002) J Neurosci 22: 6639-6649). Another group has reported that the mouse SN contains a small number of TH-expressing cells that could be labeled with BrdU, and which were thought to originate in the SVZ because they could be labeled by intraventricular injection of the fluorescent tracer dye, 1,1′-dioctadecyl-3,3,3′,3′-tetramethylindocarbocyanine perchlorate (DiI) (Zhao et al. (2003) Proc. Natl. Acad. Sci., USA, 100:7925-7930). However, others reported that TH and BrdU were present in adjacent rather than the same SN cells, and that DiI reached the SN by retrograde transport along the nigrostriatal tract (Frielingsdorf et al. (2004) Proc. Natl. Acad. Sci., USA, 101: 10177-10182).

Conflicting results have also been obtained regarding whether animal models of PD, like animal models of other neurodegenerative disorders, stimulate endogenous neurogenesis. After unilateral injection of 6-hydroxydopamine (6-OHDA) into the rat medial forebrain bundle, no BrdU-positive cells that expressed βIII-tubulin, NeuN or TH could be detected (Lie et al. (2002) J Neurosci 22:6639-6649). In contrast, administration of 6-OHDA into the SN and ventral tegmental area combined with intrastriatal infusion of growth factor-α (TGF-α) led to expansion of the EGF receptor-positive cell population in the rat SVZ and migration of these cells toward the site of TGF-α infusion (Fallon et al. (2000) Proc. Natl. Acad. Sci., USA, 97:14686-14691). Moreover, some of these migrating cells could be labeled with BrdU and expressed immature (βIII-tubulin, doublecortin) or dopaminergic (TH, DA transporter) neuronal markers. Following a single subcutaneous dose of MPTP, the number of TH-positive nigral cells that incorporated BrdU was reported to be increased (Zhao et al. (2003) Proc. Natl. Acad. Sci., USA, 100:7925-7930). Nevertheless, no such change was observed when 6-OHDA was injected into the MFB with or without concomitant administration BDNF (Frielingsdorf et al. (2004) Proc. Natl. Acad. Sci., USA, 101:10177-10182) or TGF-α (Cooper and Isacson (2004) J Neurosci 24:8924-8931) and the previous result was attributed to failure to distinguish adjacent BrdU-positive and TH-positive cells by 3-dimensional confocal analysis (Frielingsdorf et al. (2004) Proc. Natl. Acad. Sci., USA, 101:10177-10182).

In some respects, our results also contrast with previous findings using the MPTP model (Zhao et al. (2003) Proc. Natl. Acad. Sci., USA, 100:7925-7930). In our study, most BrdU-labeled cells in the SN failed to express markers for microglia, astrocytes, or neurons, although some expressed the neuronal lineage marker PSA-NCAM. These findings suggest that only limited neurogenesis may occur in SN of acutely MPTP-treated mice. Therefore, if significant injury-induced neurogenesis occurs following acute MPTP administration, it must occur outside SN (e.g. in the SVZ), and the new neurons produced either fail to migrate to the SN, or do so too slowly to be detected within the time course over which these experiments were conducted.

Our results indicate that, in contrast to the absence of evidence for large-scale neurogenesis in the SN, MPTP-induced neurogenesis contributes new neurons to the striatum. These could arise locally, or could migrate from elsewhere, such as SVZ. This resembles findings in a mouse model of HD (Ellerby et al. (2005) Proc. Natl. Acad. Sci., USA, 102: 18189-18194), although the primary sites of pathology are different in the two disorders. In fact, our observations in acutely MPTP-treated mice are reminiscent of neurotransplantation strategies for PD in which the new cells are placed in the striatum rather than SN. Perhaps in both cases, the function of lost nigrostriatal cells can be restored, at least partly, by intrastriatal substitutes. In other neurodegenerative disorders, including stroke, Alzheimer's disease and HD, neurogenesis is associated with directed migration to the site of injury. In PD, however, the new cells seem to be directed elsewhere (to the striatum rather than the SN). Perhaps this indicates that it is the degenerating nigrostriatal nerve terminals, situated in the striatum, which provide direction to newborn SVZ neurons. A neurogenesis signal emanating from degenerating nerve terminals would be consistent with the earlier involvement of terminals than somata in MPTP toxicity (Kay and Blum (2000) Dev Neurosci 22:56-67). The idea that nerve-terminal rather than cell-body dysfunction might be the driving force for injury-directed neuromigration is also consistent with findings in a mouse model of Alzheimer's disease (Jin et al. (2004) Proc. Natl. Acad. Sci., USA, 101:13363-13367). There, increased neurogenesis is observed early in the course of the disease, when synaptic dysfunction and synaptic loss are present, but cell death cannot be demonstrated. In that disorder, too, it may be affected nerve terminals rather than cell bodies that provide the stimulus for neurogenesis and the migrational target for newborn neurons.

We found previously that basal neurogenesis was not appreciably altered in R6/2 HD transgenic mice, but that if these mice were treated with FGF2, the number of new neurons in the affected striatum was increased about five-fold more than in FGF2-treated wild type mice (Ellerby et al. (2005) Proc. Natl. Acad. Sci., USA, 102: 18189-18194). Thus, the R6/2 HD mutation affects neurogenesis differently than do models of stroke (Jin et al. (2001) Proc. Natl. Acad. Sci., USA, 98:4710-4715) or AD (Jin et al. (2004) Proc. Natl. Acad. Sci., USA, 101:13363-13367), in which basal neurogenesis is increased. In this respect, mouse models of HD and PD are similar. Another similarity is that in both cases, FGF2 stimulates neurogenesis at the principal site of cell loss. FGF2 is expressed in both striatal and nigral neurons (Gonzalez et al. (1995) Brain Res 701:201-226), and loss of either could therefore produce a state of local FGF2 deficiency that precludes a neurogenesis response to injury. This would be consistent with the finding that stroke-induced neurogenesis is reduced in FGF2 knock-out mice and restored by intracerebroventricular administration of an FGF2-expressing herpes simplex virus amplicon vector (Yoshimura et al. (2001) Proc. Natl. Acad. Sci., USA, 98:5874-5879). In fact, FGF2 is depleted from SN in PD (Tooyama et al. (1993) Neurology 43:372-376) and treatment with FGF2 enhances histological and biochemical recovery from MPTP lesioning in mice (Date et al. (1993) Brain Res 621:150-154).

We conclude that, because new neurons were found at the principal site of MPTP-induced neuronal loss (SN) and in the major region to which these neurons normally project (striatum), increased neurogenesis in this model may represent a mechanism directed toward the replacement of dead or damaged neurons. If so, measures that further stimulate neurogenesis, such as the administration of neurogenesis-promoting drugs or growth factors, might have therapeutic potential in patients with PD.

### Example 3

### Mediators of MPTP-Induced Neurogenesis: FGF2

To test whether FGF2 can stimulate neurogenesis in vivo, FGF2 was interperotineally injected for 10 d and BrdU was injected intraperitoneally for 14 d following acute MPTP administration, and animals were killed 1 week later. Brain sections from SN of FGF-2- and BrdU-treated mice were immunostained one week after the last MPTP injection for BrdU and for markers of mature and immature neurons. These triple-label studies showed that BrdU-immunopositive cells co-expressed Dcx in the SN suggesting that FGF-2 can direct newborn cells in the rostral subventricular zone to the primary site of MPTP-induced injury, the SN (see, e.g., FIG. 11).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of promoting neurogenesis, treating a mammal with Huntington's disease the method comprising:
administering FGF2 or an FGF2 mutein to the mammal in an amount sufficient to ameliorate at least one symptom of the disease.

2. The method of claim 1, wherein the method consists essentially of administering FGF2.

3. The method of claim 2, wherein the FGF2 is a recombinantly expressed FGF2.

4. The method of claim 2, wherein the FGF2 is an isolated FGF2.

5. The method of claim 2, wherein the FGF2 is a human FGF2.

6. The method of claim 1, wherein the method consists essentially of administering an FGF2 mutein.

7. The method of claim 6, wherein the FGF2 mutein is a cysteine depleted FGF2 mutein.

8-11. (canceled)

12. The method of claim 1, wherein the administration is systemic.

13. The method of claim 1, wherein the administration is to the brain.

14. The method of claim 1, wherein the administration is subcutaneous.

15-17. (canceled)

18. A method of promoting neurogenesis, neuroprotection survival in a mammal with Huntington's disease, method comprising:
upregulating expression or availability of endogenous fibroblast growth factor 2 (FGF2) in the mammal.

19-21. (canceled)

22. The method of claim 18, wherein the FGF2 expression is increased by radiation treatment.

23. The method of claim 18, wherein the FGF2 expression is increased by treatment with an antidepressant.

24. The method of claim 18, wherein the FGF2 expression is increased by a ⊖2-adrenergic receptor agonist.

25-28. (canceled)

29. A method of promoting survival of a mammal with Huntington's disease, the method comprising administering FGF2 or an FGF2 mutein to the mammal in an amount sufficient to promote survival of the mammal.

30. The method of claim 29, wherein the method consists essentially of administering recombinant FGF2.

31. The method of claim 29, wherein the method consists essentially of administering a human FGF2.

32. The method of claim 29, wherein the method consists essentially of administering an FGF2 mutein.

33. The method of claim 32, wherein the FGF mutein is a cysteine-depleted FGF2 mutein.

34. The method of claim 29, wherein the administration is of a dosage of from about 3 mg/kg/day to about 15.0 mg/kg/day.

35. The method of claim 29, wherein the administration is of a dosage of from about 10 mg/kg/day to about 50 mg/kg/day.

36. The method of claim 29, wherein the administration is systemic.

37. The method of claim 29, wherein the administration is parenteral.

38. The method of claim 37, wherein the administration is to the brain.

39. The method of claim 37, wherein the administration is subcutaneous.

40. The method of claim 37, wherein the administration is by inhalation.

41. The method of claim 1, wherein the administration is of a dosage of from about 3 mg/kg/day to about 15.0 mg/kg/day.

42. The method of claim 1, wherein the administration is of a dosage of from about 10 mg/kg/day to about 50 mg/kg/day.

43. The method of claim 1, wherein the administration is parenteral.

44. The method of claim 43, wherein the administration is by inhalation.
